(19) United States
(12) Patent Application Publication
(10) Pub. No.: US 2024/0165126 A1
(43) Pub. Date: May 23, 2024

(54) TIANEPTINE OXALATE AND NALOXONE COMBINATION FOR THE TREATMENT OF MAJOR DEPRESSIVE DISORDER

(71) Applicants: TONIX PHARMACEUTICALS HOLDING CORP., Chatham, NJ (US); TONIX PHARMA LIMITED, Dubin (IE)

(72) Inventors: Seth Lederman, South Dartmouth, MA (US); Siobhan J. Fogarty, Dublin (IE)

(21) Appl. No.: 18/282,285

(22) PCT Filed: Mar. 15, 2022
(86) PCT No.: PCT/US2022/020406

§ 371 Date: Sep. 15, 2023

Related U.S. Application Data
(60) Provisional application No. 63/161,441, filed on Mar. 15, 2021.

(51) Int. Cl. A61K 31/554; A61K 9/20; A61K 31/485

(52) U.S. Cl. CPC A61K 31/554; A61K 9/2054; A61K 31/485

(57) ABSTRACT

A composition comprising tianeptine, pharmaceutically acceptable salts thereof or polymorphs of one or both and naloxone or a pharmaceutically acceptable salt thereof, methods of manufacturing the composition, and methods for preventing or treating major depressive disorder (MDD) or symptoms associated therewith comprising administering the composition to a subject in need or at risk thereof.

### CROSS-REFERENCE TO RELATED APPLICATIONS

This application claims priority and benefit from U.S. Provisional Application No. 63/161,441, filed Mar. 15, 2021, the contents of which are hereby incorporated by reference in its entirety.

### BACKGROUND

Tianeptine, or 7-[(3-chloro-6-methyl-5,5-dioxo-11H-benzo[c][2,1]benzothiazepin-11-yl)amino]heptanoic acid, is an antidepressant with cognitive restorative effects. Tianeptine affects serotonin reuptake, modulates glutamate, and activates μ-opioid receptors. Investigators have reported that it can be used to treat post-traumatic stress disorder (PTSD) (Onder E. et al., (2005), European Psychiatry 21:174-179). Tianeptine oxalate is an oxalate salt of (RS)-7-(3-chloro-6-methyl-6,11-dihydrodibenzo[c,f] [1,2]thiazepin-11-ylamino)heptanoic acid S,S-dioxide (tianeptine) and polymorphs thereof as disclosed herein. See also WO 2018/122606.

Tianeptine is not an FDA approved drug in the United States. To date, tianeptine is approved in some countries in Europe, Asia, and South America. For example, France approved tianeptine (e.g., Stablon) in 1989 as an antidepressant at a dose of 12.5 mg, three times daily. The Center for Disease Control and Prevention (CDC) published a Morbidity and Mortality Weekly Report (MMWR) in August 2018 describing incidences of tianeptine overdose from over-the-counter products and issued a warning of public health risk. Tianeptine became a scheduled drug when overdoses (e.g., 300 to 3,000 mg) and abuse (via oral, intravenous and other routes) became apparent. Common adverse events for tianeptine include anorexia, nightmares, insomnia, somnolence, dizziness, headache, tachycardia, dyspnea, gastrointestinal distress, myalgia, and asthenia.

Naloxone is an opioid receptor antagonist that is approved for use by injection (e.g., Evzio). It is also available as a nasal spray and for intravenous infusion, intramuscular injection, subcutaneous injection or intravenous injection. It is marketed for acute indications (e.g., opioid overdose, septic shock, and reversal of anesthetic agents) and used, in combination with drugs having abuse potential, for the treatment of acute pain (e.g., buprenorphine/naloxone dose 0.5 mg to 3 mg and pentazocine/naloxone dose 0.5 mg). Common adverse events for naloxone alone include vomiting, nausea, convulsion, agitation, hypertension, hypotension, and respiratory depression. The two FDA-approved products for oral chronic-use of naloxone include Talwin Nx® (3-6 mg/day naloxone) and Targiniq ER® (10-40 mg/day naloxone HCl). Other products containing naloxone, or a naloxone derivative, that are FDA-approved for chronic use include sublingual Suboxone® (buprenorphine and naloxone for maintenance treatment of opioid dependence) and oral PEGylated naloxone Movantik® (recommended for opioid-induced constipation).

An estimated 17-18 million adults in the United States have experienced at least one major depressive episode, which represents about 7% of all adults (age 18 or older) in the United States. The prevalence of a major depressive episode is higher among adult females (e.g., about 8.7%) compared to males (e.g., about 5.3%). In addition, the prevalence of a major depressive episode was highest among individuals aged 18-25 (e.g., about 13.1%). Major Depressive Disorder (MDD) is a leading cause of disability worldwide and is a mood disorder that can persist for 2 or more weeks. MDD affects how a person feels, thinks, and handles daily activities and can significantly impair all aspects of life as patients present with persistent sadness or anxiousness, irritability, anhedonia, significant weight change, appetite disturbances, sleep disturbances, psychomotor agitation or retardation, fatigue or loss of energy, feelings of worthlessness, diminished ability to think or concentrate, indecisiveness, aches or pains without a clear physical cause, or recurrent thoughts of death or suicide.

The current mainstays of pharmacological treatments for depression include the use of selective serotonin reuptake inhibitors (SSRIs) (Fava et al., 2007; Zupancic et al., 2006) and serotonin-norepinephrine reuptake inhibitors (SNRIs) (Stahl et al., 2005), however, these therapies are not effective in all patients, and are often associated with undesirable side effects, such as weight gain and sexual dysfunction. Even with multiple consecutive treatments, only a small proportion of patients remain asymptomatic (Rush, 2007). Opioids and narcotic painkillers (e.g., oxycodone, hydrocodone, fentanyl, and tramadol) are also prescribed to treat MDD but there is an increased risk of long-term use, abuse (e.g., parenteral, transmucosal, intranasal or oral drug abuse) and addiction. There is, therefore, an unmet need to safely treat MDD. A composition of tianeptine, pharmaceutically acceptable salts thereof and polymorphs of one or both and naloxone and pharmaceutically acceptable salts thereof, as described in various embodiments of this disclosure, and methods of using those compositions meet this unmet need and improve symptoms associated with Major Depressive Disorder, while at the same time minimizing potential for drug abuse.

### SUMMARY OF THIS DISCLOSURE

In a first aspect, the present disclosure provides a composition comprising tianeptine or a pharmaceutically acceptable salt thereof or polymorphs of one or both, and naloxone or a pharmaceutically acceptable salt thereof.

In some embodiments of the compositions of this disclosure, the pharmaceutically acceptable salt of the tianeptine is tianeptine oxalate or tianeptine sodium or polymorphs thereof. In some embodiments of the compositions of this disclosure, the pharmaceutically acceptable salt of tianeptine is tianeptine oxalate or polymorphs thereof. In some embodiments of the compositions of this disclosure, the pharmaceutically acceptable salt of tianeptine is tianeptine oxalate. In some embodiments of the compositions of this disclosure, the tianeptine oxalate is an anhydrous crystalline hemi-oxalate salt, an anhydrous crystalline mono-oxalate salt, a mixture thereof or polymorphs of any of them. In some embodiments, the compositions of this disclosure comprise two or more layers of tianeptine oxalate, each layer comprising one or more of an anhydrous crystalline hemi-oxalate salt, an anhydrous crystalline mono-oxalate salt, a mixture thereof or polymorphs of any of them.

In some embodiments of the compositions of this disclosure, the tianeptine oxalate is an anhydrous crystalline hemi-oxalate salt or polymorphs thereof. In some embodiments of the compositions of this disclosure, the tianeptine oxalate is an anhydrous crystalline hemi-oxalate salt that exhibits an X-ray diffraction pattern (XRPD) comprising at least one peak selected from the group consisting of 8.2, 8.6, 9.1, and 9.5 degrees 2θ±0.3 degrees 2θ. In some embodiments of the compositions of this disclosure, the anhydrous crystalline tianeptine hemi-oxalate salt exhibits an XRPD pattern comprising at least one peak selected from the group consisting of 8.2, 8.6, 9.1, and 9.5 degrees 2θ±0.3 degrees 2θ and at least one additional peak selected from the group consisting of 4.5, 11.5, 14.2, 15.2, 15.8, 16.2, 19.2, 22.1, 23.9, 26.9, and 27.4 degrees 2θ±0.3 degrees 2θ.

In some embodiments of the compositions of this disclosure, the tianeptine oxalate is an anhydrous crystalline mono-oxalate salt or polymorphs thereof. In some embodiments of the compositions of this disclosure, the tianeptine oxalate is an anhydrous crystalline mono-oxalate salt that exhibits an X-ray diffraction pattern (XRPD) comprising at least one peak selected from the group consisting of 10.1 and 10.5 degrees 2θ±0.3 degrees 2θ. In some embodiments of the compositions of this disclosure, the anhydrous crystalline tianeptine mono-oxalate salt exhibits an XRPD pattern comprising at least one peak selected from the group consisting of 10.1 and 10.5 degrees 2θ±0.3 degrees and at least one additional peak selected from the group consisting of 7.5, 8.3, 11.9, 14.7, 16.2, 16.3, 17.9, 18.7, 21.0, 21.7, and 22.1 degrees 2θ±0.3 degrees 2θ.

In some embodiments of the compositions of this disclosure, the tianeptine oxalate is a mixture of an anhydrous crystalline hemi-oxalate salt and an anhydrous crystalline mono-oxalate salt or polymorphs thereof. In some embodiments, the mixture of the anhydrous crystalline tianeptine hemi-oxalate salt and the anhydrous crystalline tianeptine mono-oxalate salt exhibits an X-ray diffraction pattern (XRPD) comprising at least one peak selected from the group consisting of 10.1 and 10.5 degrees 2θ±0.3 degrees 2θ. In some embodiments of the compositions of this disclosure, the mixture exhibits an XRPD pattern comprising at least one peak selected from the group consist of 10.1 and 10.5 degrees 2θ±0.3 degrees 2θ and further comprising at least one additional peak selected from the group consisting of 7.5, 8.3, 11.9, 14.7, 16.2, 16.3, 17.9, 18.7, 21.0, 21.7, and 22.1 degrees 2θ±0.3 degrees 2θ.

In some embodiments of the compositions of this disclosure, the pharmaceutically acceptable salt of naloxone is an acid salt. In some embodiments of the compositions of this disclosure, the naloxone acid salt is naloxone hydrochloride. In some embodiments of the compositions of this disclosure, the naloxone hydrochloride is naloxone hydrochloride dihydrate.

In some embodiments of the compositions of this disclosure, the tianeptine, or a pharmaceutically acceptable salt thereof or a polymorph of one or both is present in an amount between 1% w/w to 30% w/w, between 5% w/w to 25% w/w, or between 10% w/w to 20% w/w. In some embodiments of the compositions of this disclosure, the tianeptine, or a pharmaceutically acceptable salt thereof or a polymorph of one or both is present in an amount equivalent to about 14.28% w/w of the tianeptine free base. In some embodiments of the compositions of this disclosure, the tianeptine hemi-oxalate salt in an amount of about 15.76% w/w.

In some embodiments of the compositions of this disclosure, the naloxone or a pharmaceutically acceptable salt thereof is present in an amount between 0.01% w/w to 5% w/w or between 0.1% w/w to 2% w/w. In some embodiments of the compositions of this disclosure, the naloxone or a pharmaceutically acceptable salt thereof is present in an amount of less than 1% w/w or less than 0.5% w/w. In some embodiments of the compositions of this disclosure, the naloxone or a pharmaceutically acceptable salt thereof is present in an amount equivalent to about 0.40% w/w of the naloxone free base. In some embodiments of the compositions of this disclosure, the pharmaceutically acceptable salt of naloxone is naloxone hydrochloride. In some embodiments, the naloxone hydrochloride dihydrate is present in the composition of this disclosure in an amount of about 0.49% w/w.

In some embodiments of the compositions of this disclosure, the tianeptine, a pharmaceutically acceptable salt thereof or a polymorph of one or both is present in an amount between 0.1 mg to 60 mg, between 1 mg to 50 mg, or between 10 mg to 40 mg. In some embodiments of the compositions of this disclosure, the tianeptine, a pharmaceutically acceptable salt thereof or a polymorph of one or both is present in an amount of 50 mg or less, 45 mg or less, or 40 mg or less. In some embodiments of the compositions of this disclosure, the tianeptine, a pharmaceutically acceptable salt thereof or a polymorph of one or both is present in an amount equivalent to about 35.7 mg of the tianeptine free base. In some embodiments of the compositions of this disclosure, the tianeptine hemi-oxalate salt or polymorph thereof is present in an amount of about 39.4 mg.

In some embodiments of the compositions of this disclosure, the naloxone or a pharmaceutically acceptable salt thereof is present in an amount between 0.01 mg to 10 mg, between 0.1 mg to 5 mg, or between 0.5 mg to 2 mg. In some embodiments of the compositions of this disclosure, the naloxone or a pharmaceutically acceptable salt thereof is present in an amount of 5 mg or less, or 2 mg or less. In some embodiments of the compositions of this disclosure, the naloxone or a pharmaceutically acceptable salt thereof is present in an amount equivalent to about 1 mg of the naloxone free base. In some embodiments of the compositions of this disclosure, the naloxone hydrochloride dihydrate is present in the composition in an amount of about 1.22 mg.

In some embodiments, the compositions of this disclosure further comprise one or more excipients. In some embodiments of the compositions of this disclosure, the excipient comprises a filler, a controlled release polymer, a lubricant, a glidant, a pH adjusting agent, or a buffer.

In some embodiments of the compositions of this disclosure, the filler is selected from the group consisting of microcrystalline cellulose, gelatin, sucrose, starch, acacia, tragacanth, alginic acid, cellulose, methyl cellulose, ethyl cellulose, hydroxypropyl methyl cellulose, hydroxypropyl cellulose, sodium carboxymethyl cellulose, polyvinyl pyrrolidone, polyethylene glycol, polyvinyl pyrrolidone, lactose monohydrate and cross-linked polyvinyl pyrrolidone. In some embodiments of the compositions of this disclosure, the filler is microcrystalline cellulose or lactose monohydrate. In some embodiments of the compositions of this disclosure, the filler is present in an amount between 0.5% w/w to 50% w/w.

In some embodiments of the compositions of this disclosure, the controlled release polymer is selected from the group consisting of hydroxypropyl methylcellulose, hydroxypropyl cellulose, polyvinyl pyrrolidone, carboxymethyl cellulose, methylcellulose, hydroxyethyl cellulose, ethyl hydroxyethyl cellulose, sodium carboxymethyl cellulose, sodium alginate, xanthan gum, carrageenan, chitosan, guar gum, pectin, polyethylene oxide, ethylcellulose, hypromellose acetate succinate, cellulose acetate and cellulose acetate propionate. In some embodiments of the compositions of this disclosure, the controlled release polymer is hydroxypropyl methylcellulose. In some embodiments of the compositions of this disclosure, the controlled release polymer is present in an amount between 5% w/w to 30% w/w.

In some embodiments of the compositions of this disclosure, the lubricant is selected from the group consisting of magnesium stearate, calcium stearate, zinc stearate, stearic acid, myristic acid, palmitic acid, sodium stearyl fumarate, boric acid, carbowax (PEG) 4000/6000, sodium oleate, sodium benzoate, sodium acetate, sodium lauryl sulfate, magnesium lauryl sulfate, glyceride esters, glyceryl monostearate, glyceryl tribehenate, glyceryl dibehenate, sugar esters, sorbitan monostearate, sucrose monopalmitate, waxes, sterowet, glyceryl behenate, liquid paraffin and talc. In some embodiments of the compositions of this disclosure, the lubricant is magnesium stearate. In some embodiments of the compositions of this disclosure, the lubricant is present in an amount between 0.1% w/w to 5% w/w.

In some embodiments of the compositions of this disclosure, the glidant is selected from the group consisting of starch, cornstarch, silica derivatives, fumed silica, colloidal silicon dioxide, hydrophilic fumed silica, syloid, pyrogenic silica, ascorbyl palmitate, calcium palmitate, magnesium stearate, talc and hydrated sodium sulfoaluminate. In some embodiments of the compositions of this disclosure, the glidant is colloidal silicon dioxide. In some embodiments of the compositions of this disclosure, the glidant is present in in an amount between 0.1% w/w to 2% w/w.

In some embodiments of the compositions of this disclosure, the pH adjusting agent is selected from the group consisting of citric acid monohydrate, trisodium citrate dihydrate, sodium acetate, acetic acid, tartaric acid, sodium hydrogen carbonate, potassium citrate monohydrate, sodium citrate, tromethamine (Tris), sodium hydroxide, sodium phosphate, disodium hydrogen phosphate, sodium bisphosphate, boric acid, sodium borate, benzoic acid, sodium benzoate, aspartic acid, and maleic acid. In some embodiments of the compositions of this disclosure, the pH adjusting agent is citric acid monohydrate, trisodium citrate dihydrate, or a combination thereof. In some embodiments of the compositions of this disclosure, the pH adjusting agent is present in an amount between 0.1 w/w to 2% w/w.

In some embodiments of the compositions of this disclosure, the buffer is selected from the group consisting of trisodium citrate dihydrate, citric acid, boric acid, sodium citrate, potassium bitartrate, calcium acetate, ammonium phosphate, calcium chloride, citric acid salts, lactic acid, magnesium trisilicate, phosphoric acid, potassium, potassium citrate, potassium phosphate, sodium acetate, sodium borate, sodium lactate, succinic acid, and monothioglycerol. In some embodiments of the compositions of this disclosure, the buffer is trisodium citrate dihydrate. In some embodiments of the compositions of this disclosure, the buffer is present in an amount between 0.1% w/w to 2% w/w.

In some embodiments, the compositions of this disclosure further comprise a pharmaceutically acceptable carrier.

In some embodiments, the compositions of this disclosure are in the form of a tablet, a mini-tablet, a chewable tablet, an orodispersible tablet, a dissolvable tablet, a scored tablet, a coated tablet, a suppository, or a granule. In some embodiments, the compositions of this disclosure are in the form of a tablet. In some embodiments of the compositions of this disclosure, the tablet is formulated so as to be capable of immediate release, controlled release, sustained release, extended release, or slow release of one or both of the tianeptine, a pharmaceutically acceptable salt thereof or a polymorph of one or both and the naloxone or a pharmaceutically salt thereof. In some embodiments of the compositions of this disclosure, the tablet is formulated so as to be capable of controlled release of the tianeptine, a pharmaceutically acceptable salt thereof or a polymorph of one or both and the naloxone or a pharmaceutically acceptable salt thereof. In some embodiments of the compositions of this disclosure, the tablet is formulated so as to be capable of controlled release of tianeptine oxalate or a polymorph thereof and naloxone hydrochloride. In some embodiments, the tianeptine oxalate is an anhydrous crystalline hemi-oxalate salt and wherein the naloxone hydrochloride is naloxone hydrochloride dihydrate. In some embodiments of the compositions of this disclosure, the tablet is formulated as to be capable of controlled release of an anhydrous crystalline hemi-oxalate salt of the tianeptine and naloxone hydrochloride dihydrate.

In some embodiments of the compositions of this disclosure, the tablet is characterized by a hardness of greater than 10 Kp. In some embodiments of the compositions of this disclosure, the tablet is characterized by a tensile strength of greater than 2 MPa.

In another aspect, the present disclosure provides methods of preventing and/or treating major depressive disorder (MDD) or symptoms associated therewith, comprising administering to a subject in need thereof or at risk thereof, a composition of this disclosure comprising one or more of tianeptine or a pharmaceutically acceptable salt thereof or polymorphs of one or both, and naloxone or a pharmaceutically acceptable salt thereof. In some embodiments, the method of this disclosure comprises administering a tianeptine hemi-oxalate salt, a tianeptine mono-oxalate salt or a mixture of them, and naloxone or a pharmaceutically acceptable salt thereof. In some embodiments, the method of this disclosure comprises administering a tianeptine hemi-oxalate salt and naloxone or a pharmaceutically acceptable salt thereof. In some embodiments, the method of this disclosure comprises administering a tianeptine mono-oxalate salt and naloxone or a pharmaceutically acceptable salt thereof. In some embodiments, the method of this disclosure comprises administering a mixture of a tianeptine hemi-oxalate salt and a tianeptine mono-oxalate salt, and naloxone or a pharmaceutically acceptable salt thereof.

In some embodiments, the methods of this disclosure comprise administering a composition of this disclosure one or more times daily. In some embodiments, the methods of this disclosure comprise administering the composition of this disclosure once daily. In some embodiments, the methods of this disclosure comprise administering a composition of this disclosure orally, sublingually, buccally, palatially, rectally, or vaginally. In some embodiments, the methods of this disclosure comprise administering a composition of this disclosure orally.

In another aspect, the present disclosure provides methods of manufacturing a composition of this disclosure comprising tianeptine or a pharmaceutically acceptable salt thereof or a polymorph of one or both, and naloxone or a pharmaceutically acceptable salt thereof. In some embodiments, the method comprises manufacturing a composition comprising a tianeptine hemi-oxalate salt, or a tianeptine mono-oxalate salt or a mixture of them and naloxone or a pharmaceutically acceptable salt thereof. In some embodiments, the method comprises manufacturing a composition comprising a tianeptine hemi-oxalate salt and naloxone or a pharmaceutically acceptable salt thereof. In some embodiments, the method comprises manufacturing a composition comprising a tianeptine mono-oxalate salt and naloxone or a pharmaceutically acceptable salt thereof. In some embodiments, the method comprises manufacturing a composition comprising a mixture of a tianeptine hemi-oxalate salt and a mono-oxalate salt, and naloxone or a pharmaceutically acceptable salt thereof.

In some embodiments of the methods of manufacturing of this disclosure, the naloxone or a pharmaceutically acceptable salt thereof is mixed with a pH adjusting agent prior to its mixing with the tianeptine, a pharmaceutically acceptable salt thereof or a polymorph of one or both, and one or more excipients. In some embodiments of the methods of manufacturing of this disclosure, the naloxone or a pharmaceutically acceptable salt thereof, before mixing with the tianeptine, a pharmaceutically salt thereof or a polymorph of one or both, is comprised of particles that are less than 20 μm.

In some embodiments of the methods of manufacturing of this disclosure, the composition is compressed to form a tablet. In some embodiments of the methods of manufacturing of this disclosure, the tablet is compressed to a hardness of greater than 10 Kp. In some embodiments of the methods of manufacturing of this disclosure, the tablet is compressed to a tensile strength of greater than 2 MPa.

In some embodiments the compositions of this disclosure prevents or reduces the potential for abuse of the tianeptine, the pharmaceutically acceptable salt thereof or the polymorph of one or both.

### DETAILED DESCRIPTION

In various embodiments, the present disclosure provides compositions and methods for treating major depressive disorder or one or more symptoms associated therewith in a subject in need or at risk thereof, while at the same time minimizing the potential for drug abuse. In various embodiments, the composition comprises tianeptine, pharmaceutically acceptable salts (e.g., oxalate) thereof, or polymorphs of one or both and naloxone or pharmaceutically acceptable salts thereof.

### Definitions

Unless otherwise defined herein, scientific and technical terms used in this application shall have the meanings that are commonly understood by those of ordinary skill in the art. In case of conflict, the present specification, including definitions, will control.

It should be understood that any of the embodiments described herein, including those described under different aspects of the disclosure and different parts of the specification (including embodiments described only in the Examples) can be combined with one or more other embodiments of this disclosure, unless explicitly disclaimed or improper. Combination of embodiments are not limited to those specific combinations described in the multiple dependent embodiments of this disclosure.

All of the publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The term “including,” as used herein, means “including but not limited to.” “Including” and “including but not limited to” are used interchangeably. Thus, these terms will be understood to imply the inclusion of a stated integer (or components) or group of integers (or components), but not the exclusion of any other integer (or components) or group of integers (or components).

Any example(s) following the term “e.g.” or “for example” is not meant to be exhaustive or limiting.

Unless otherwise required by context, singular terms shall include pluralities and plural terms shall include the singular.

The articles “a”, “an” and “the” are used herein to refer to one or to more than one (i.e., to at least one) of the grammatical object of the article.

Notwithstanding that the disclosed numerical ranges and parameters are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10. As used herein, the term “w/w” permits a variation of ±10% w/w, as apparent from the context, within the range of the significant digit, e.g., at the lower end of a range 10% w/w includes 9% w/w, and at the upper end of a range 30% w/w includes 33% w/w. As used herein, the term “mg” permits a variation of 5% mg, as apparent from the context, within the range of the significant digit, e.g., at the lower end of a range 10 mg includes 9.5 mg, and at the upper end of a range 10 mg includes 10.5 mg.

Where aspects or embodiments are described in terms of a Markush group or other grouping of alternatives, the present application encompasses not only the entire group listed as a whole, but each member of the group individually and all possible subgroups of the main group, and also the main group absent one or more of the group members.

Exemplary methods and materials are described herein, although methods and materials similar or equivalent to those described herein can also be used in the practice or testing of the various aspects and embodiments. The materials, methods, and examples are illustrative only and not intended to be limiting.

In order that the disclosure may be more readily understood, certain terms are first defined. These definitions should be read in light of the remainder of the disclosure as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, the term “treat” and its cognates refer to a full or partial amelioration or modulation of major depressive order or at least one discernible symptom associated therewith. In some embodiments, “treat at least one discernible symptom” refers to a reduction of one or more of persistent sadness, anxiousness, or “empty” mood associated with major depressive disorder. In some embodiments, “treat at least one discernible symptom” refers to a reduction of feeling hopeless and/or pessimism associated with major depressive disorder. In some embodiments, “treat of at least one discernible symptom” refers to reduction of irritability associated with major depressive disorder. In some embodiments, “treat” refers to a reduction of one or more of feeling guilt, worthlessness, or helplessness associated with major depressive disorder. In some embodiments, “treat at least one discernible symptom” refers to an increased feeling of interest or pleasure in hobbies and/or activities (e.g., reduction of anhedonia) associated with major depressive disorder. In some embodiments, “treat at least one discernible symptom” refers to a reduction of fatigue or to an increase in energy associated with major depressive disorder. In some embodiments, “treat at least one discernible symptom” refers to a reduction of slow movement and/or speech associated with major depressive disorder. In some embodiments, “treat at least one discernible symptom” refers to a reduction of psychomotor agitation and/or retardation associated with major depressive disorder. In some embodiments, “treat at least one discernible symptom” refers to a reduction of feeling restless and/or having trouble sitting still associated with major depressive disorder. In some embodiments, “treat at least one discernible symptom” refers to a reduction of in one or more of difficulty concentrating, remembering, or making decisions associated with major depressive disorder. In some embodiments, “treat at least one discernible symptom” refers to a reduction in one or more of difficulty sleeping, early-morning awakening, or oversleeping (e.g., reduction of insomnia or hypersomnia) associated with major depressive disorder. In some embodiments, “treat at least one discernible symptom” refers to improvement of appetite and/or reduction of body weight change associated with major depressive disorder. In some embodiments, “treat at least one discernible symptom” refers to a reduction of thoughts of death and/or suicide associated with major depressive disorder. In some embodiments, “treat at least one discernible symptom” refers to a reduction of or no suicide attempts associated with major depressive disorder. In some embodiments, “treat at least one discernible symptom” refers to a reduction in one or more of aches or pains, headaches, cramps, or digestive problems without a clear physical cause associated with major depressive disorder.

As used herein, the term “about” modifying the quantity of an ingredient, parameter, calculation, or measurement in the compositions of this disclosure refers to the variation in the numerical quantity that can occur, for example, through typical measuring and liquid handling procedures used for making compositions or pharmaceutical compositions in the real world; through inadvertent error in these procedures; through differences in the manufacture, source, or purity of the ingredients employed to make the compositions or carry out the methods; and the like without having a substantial effect on the chemical or physical attributes of the compositions or methods of the disclosure. Such variation can be within an order of magnitude, typically within 10%, more typically within 5%, of a given value or range. The term “about” also encompasses amounts that differ due to different equilibrium conditions for a composition resulting from a particular initial mixture. Reference to “about” a value or parameter herein includes (and describes) in particular embodiments that are directed to that value or parameter per se. For example, description referring to “about X” includes description of “X.” Numeric ranges are inclusive of the numbers defining the range. Whether or not modified by the term “about,” the paragraphs include equivalents to the quantities.

As used herein, “subject” and “patient” are used interchangeably herein and refer to mammals including, but not limited to, human and non-human animals. These terms include mammals, such as humans, and primates (e.g., monkey). In some embodiments, the subject is a human. Accordingly, the term “subject” or “patient” as used herein means any mammalian patient or subject to which the compositions of the disclosure may be administered. In some embodiments, the subject is in need of treatment of major depressive disorder (MDD).

As used herein, “tianeptine” refers to tianeptine free base.

As used herein, “naloxone” refers to naloxone free base.

As used herein, “tianeptine oxalate” refers to a tianeptine oxalate salt and/or polymorph. The tianeptine oxalate salts can take several forms including, but not limited to, hydrates and solvates. Other forms of tianeptine oxalate salts include, but are not limited to, polymorphs. Examples of tianeptine oxalate salts include tianeptine hemi-oxalate and tianeptine mono-oxalate (e.g., Form A or Form B) as described in WO 2018/122606.

As used herein, the term “major depressive episode” (MDE) refers to a period characterized by symptoms of major depressive disorder. In some embodiments, an MDE is assessed by the Mini International Neuropsychiatric Interview, Version 7.0.2 (M.I.N.I 7.0.2).

As used herein, the term “drug abuse” refers to an excessive administration of a psychoactive drug that can lead to physical, social or emotional harm. In some embodiments, the “drug abuse” is by excessive parenteral administration (e.g., intravenous injection, subcutaneous injection, intramuscular injection, inhalation and insufflation). In some embodiments, the “drug abuse” is due to excessive oral administration. In some embodiments, the psychoactive drug includes tianeptine, its pharmaceutically acceptable salts, (e.g., oxalate salts) thereof and polymorphs of one or both.

As used herein, the term “filler”, also known as “binder”, refers to an agent or substance that is believed to promote cohesiveness in a mixture. Non-limiting examples of fillers, useful in various embodiments of this disclosure, include microcrystalline cellulose (e.g., microcrystalline cellulose PH 101 and microcrystalline cellulose PH 102), gelatin, sucrose, starch, acacia, tragacanth, alginic acid, cellulose, methyl cellulose, ethyl cellulose, hydroxypropyl methyl cellulose, hydroxypropyl cellulose, sodium carboxymethyl cellulose, polyvinyl pyrrolidone, polyethylene glycol, polyvinyl pyrrolidone, lactose monohydrate (e.g., spray-dried lactose monohydrate) and cross-linked polyvinyl pyrrolidone.

As used herein, the term “controlled release polymer” refers to a polymer that enables a formulation that is capable of controlling the release rate of an active agent from a polymer matrix. In some embodiments, the agent is encapsulated or conjugated with the polymer. In some embodiments, the controlled release polymer allows for controlled and/or continuous release of the agent. Non-limiting examples of controlled release polymers, useful in various embodiments of this disclosure, include hydroxypropyl methylcellulose (e.g., HPMC K4 and HPMC K100LV), hydroxypropyl cellulose, polyvinyl pyrrolidone, carboxymethyl cellulose, methylcellulose, hydroxyethyl cellulose, ethyl hydroxyethyl cellulose, sodium carboxymethyl cellulose, sodium alginate, xanthan gum, carrageenan, chitosan, guar gum, pectin, polyethylene oxide, ethylcellulose, hypromellose acetate succinate, cellulose acetate and cellulose acetate propionate.

As used herein, the term “lubricant” refers to a substance or agent that is believed to reduce friction during tablet ejection. In some embodiments, the lubricant prevents adhesion of the tablet. In some embodiments, the lubricant reduces interparticulate friction. In some embodiments, the lubricant is a detergent. In some embodiments, the lubricant prevents the separation of tianeptine oxalate and naloxone or a pharmaceutically acceptable salt thereof in a composition of this disclosure. Non-limiting examples of lubricants, useful in various embodiments of this disclosure, include magnesium stearate (e.g., LIGAMED MF-2-V), calcium stearate, zinc stearate, stearic acid, myristic acid, palmitic acid, sodium stearyl fumarate, boric acid, carbowax (PEG) 4000/6000, sodium oleate, sodium benzoate, sodium acetate, sodium lauryl sulfate, magnesium lauryl sulfate, glyceride esters, glyceryl monostearate, glyceryl tribehenate, glyceryl dibehenate, sugar esters, sorbitan monostearate, sucrose monopalmitate, waxes, sterowet, glyceryl behenate, liquid paraffin and talc.

As used herein, the term “glidant” refers to a substance or agent that is believed to enhance flow property or improves flowability of a powder. Non-limiting examples of glidants, useful in various embodiments of this disclosure, include starch, cornstarch, silica derivatives, colloidal silicon dioxide (e.g., hydrophilic fumed silica), syloid, pyrogenic silica (also known as fumed silica), ascorbyl palmitate, calcium palmitate, magnesium stearate, talc and hydrated sodium sulfoaluminate.

As used herein, the term “pH adjusting agent” refers to an agent or combination of them that is believed to alter or control the acidity or alkalinity of a composition. Non-limiting examples of pH adjusting agents, useful in various embodiments of this disclosure, include citric acid monohydrate, trisodium citrate dihydrate, sodium acetate, acetic acid, tartaric acid, sodium hydrogen carbonate, potassium citrate monohydrate, sodium citrate, tromethamine (Tris), sodium hydroxide, sodium phosphate, disodium hydrogen phosphate, sodium bisphosphate, boric acid, sodium borate, benzoic acid, sodium benzoate, aspartic acid, and maleic acid.

As used herein, the term “buffer” refers to a compound that is believed to resist change in pH upon addition of an acid or base. Non-limiting examples of buffers include trisodium citrate dihydrate, citric acid, boric acid, sodium citrate, potassium bitartrate, calcium acetate, ammonium phosphate, calcium chloride, citric acid salts, lactic acid, magnesium trisilicate, phosphoric acid, potassium, potassium citrate, potassium phosphate, sodium acetate, sodium borate, sodium lactate, succinic acid, and monothioglycerol.

As used herein, the term “tₘₐₓ” refers to the time after dosing at which the maximum concentration is observed.

As used herein, the term “Cₘₐₓ” refers to the maximum observed concentration as measured after dosing.

As used herein, the term “Cₘₐₓ/Dose” refers to the Cₘₐₓ divided by the dose that is administered.

As used herein, the term “AUCₜₗₐₛₜ” refers to the area under the concentration versus time curve from the start of dose administration to the last quantifiable concentration that is observed calculated using the linear trapezoidal method.

As used herein, the term “AUCₜₗₐₛₜ/Dose” refers to the AUCₜₗₐₛₜ divided by the dose that is administered.

As used herein, the term “AUC₍₀₋ₜ₎” refers to the area under the concentration time curve from time zero to t, where t denotes a specific sampling time following dosing.

As used herein, the term “AUC₍₀₋ₜ₎/Dose” refers to the AUC₍₀₋ₜ₎ normalized for dose that is administered.

As used herein, the term “tₗₐₛₜ” refers to the time after dosing at which the last quantifiable concentration is observed.

As used herein, the term “R_{AUC}” refers to the area under the curve from T1 to T2 after repeat dosing divided by the area under the curve from T1 to T2 during the initial dosing interval at which the composition is administered.

As used herein, the term “naloxone impurities” refers to a degradant of naloxone or a pharmaceutically acceptable salt thereof. In some embodiments, the degradant of naloxone (e.g., naloxone N-oxide) is due to oxidation of naloxone. In some embodiments, the naloxone impurities are less than 0.60% w/w in the composition of this disclosure. In some embodiments, the naloxone impurities are less than 0.55% w/w in the composition of this disclosure. In some embodiments, the naloxone impurities are less than 0.50% w/w in the composition of this disclosure.

### Major Depressive Disorder

Major depressive disorder (MDD), also referred to as “clinical depression,” is a mood disorder in which symptoms that affect how a person feels, thinks, and handles daily activities are present almost every day for at least two weeks.

Symptoms associated with MDD include (1) persistent sad, anxious, or “empty” mood; (2) feeling of hopelessness, or pessimism; (3) irritability; (4) feelings of guilt, worthlessness, or helplessness; (5) loss of interest or pleasure in hobbies and activities (e.g., anhedonia); (6) decreased energy or fatigue; (7) moving or talking more slowly; (8) psychomotor agitation or retardation; (9) feeling restless or having trouble sitting still; (10) difficulty concentrating, remembering, or making decisions; (11) difficulty sleeping, early-morning awakening, or oversleeping (e.g., insomnia or hypersomnia); (12) appetite and/or weight changes (e.g., a change of more than 5% of body weight in a month); (13) recurring thoughts of death or suicide; (14) suicide attempts; (15) aches or pains, headaches, cramps, or digestive problems without a clear physical cause and/or that do not ease even with treatment.

MDD can be diagnosed based on (1) a physical exam; (2) laboratory tests (e.g., blood test of complete blood count and/or assessing thyroid function); (3) psychiatric evaluation (e.g., health professional asks about symptoms, thoughts, feelings and behavior patterns and/or patient fills out a questionnaire, such as the Hospital Anxiety and Depression Scale (HADS)); (4) Diagnostic and Statistical Manual of Mental Disorders (e.g., DSM-5 lists criteria for depression published by the American Psychiatric Association).

DSM-5 criteria for diagnosing MDD includes at least 5 symptoms that are present during the same 2-week period, and at least one of the symptoms is diminished interest and pleasure or depressed mood. The symptoms associated with the DSM-5 criteria include (1) depressed mood (for children and adolescents, this can also be an irritable mood); (2) diminished interest or loss of pleasure in almost all activities (e.g., anhedonia); (3) significant weight change or appetite disturbance (for children, this can be failure to achieve expected weight gain); (4) sleep disturbance (e.g., insomnia or hypersomnia); (5) psychomotor agitation or retardation; (6) fatigue or loss of energy; (7) feelings of worthlessness; (8) diminished ability to think or concentrate; indecisiveness; (9) recurrent thoughts of death, recurrent suicidal ideation without a specific plan, or a suicide attempt or specific plan for committing suicide.

Any suitable route of administration may be employed for providing the subject with the compositions and dosage units of embodiments of this disclosure. For example, suitable routes of administration, including oral, sublingual, buccal, palatal, rectal, or vaginal and the like, may be employed as appropriate. In some embodiments, the dosage form is a tablet, a mini-tablet, a chewable tablet, an orodispersible tablet, dissolvable tablet, a scored tablet, a coated tablet, a suppository, or a granule.

### Tianeptine and Naloxone Metabolism

Tianeptine and naloxone have well-established metabolic pathways that are different and non-overlapping. Tianeptine has good bioavailability (e.g., about 99%) and has minimal first pass metabolism. The major metabolites of tianeptine are MC5 and MC3, which result from β-oxidation (Wagstaff et al., CNS Drugs, 15(3), 231-259 (2001)).

Naloxone has low (e.g., ≤2%) systemic bioavailability when orally administered due to extensive first pass hepatic metabolism (Smith et al., Int J Clin Pharmacol Ther, 50: 360-367 (2012)). The primary mechanism of clearance for naloxone is glucuronidation. Naloxone metabolism is primarily mediated by UGT1A8 and UGT2B7, leading mainly to 6β-naloxol, naloxone-3β-glucuronide and 6β-naloxol-3β-glucuronide. The enzymes that lead to naloxone elimination are not known to metabolize tianeptine (Targiniq ER® label, Section 13.2). Naloxone is a well-established μ-opioid antagonist and is not expected to have efficacy in MDD.

### Therapeutic Composition

In some aspects, the present disclosure provides compositions comprising tianeptine or a pharmaceutically acceptable salt thereof or polymorphs of one or both, and naloxone or a pharmaceutically acceptable salt thereof.

In some embodiments, the compositions of this disclosure comprise a pharmaceutically acceptable salt of tianeptine, wherein the pharmaceutically acceptable salt is tianeptine oxalate or tianeptine sodium or polymorphs thereof. In some embodiments, the compositions of this disclosure comprise a pharmaceutically acceptable salt of tianeptine, wherein the pharmaceutically acceptable salt is tianeptine oxalate or a polymorph thereof. In some embodiments, the compositions of this disclosure comprise a pharmaceutically acceptable salt of tianeptine, wherein the pharmaceutically acceptable salt is tianeptine sodium or a polymorph thereof. In some embodiments, the compositions of this disclosure comprise a pharmaceutically acceptable salt of tianeptine, wherein the pharmaceutically acceptable salt is tianeptine oxalate. In some embodiments, the compositions of this disclosure comprise a pharmaceutically acceptable salt of tianeptine, wherein the pharmaceutically acceptable salt is tianeptine sodium.

In some embodiments, the compositions of this disclosure comprise a tianeptine oxalate. In some embodiments of the compositions of this disclosure, the tianeptine oxalate is an anhydrous crystalline hemi-oxalate salt, an anhydrous crystalline mono-oxalate salt, a mixture thereof or polymorphs of any of them. In some embodiments, the compositions of this disclosure comprise an anhydrous crystalline hemi-oxalate tianeptine salt or a polymorph thereof. In some embodiments, the compositions of this disclosure comprise an anhydrous crystalline mono-oxalate tianeptine salt or a polymorph thereof. In some embodiments, the compositions of this disclosure comprise a mixture of an anhydrous crystalline hemi-oxalate tianeptine salt and an anhydrous crystalline mono-oxalate tianeptine salt or polymorphs thereof. In some embodiments, the compositions of this disclosure comprise an anhydrous crystalline hemi-oxalate tianeptine salt. In some embodiments, the compositions of this disclosure comprise an anhydrous crystalline mono-oxalate tianeptine salt. In some embodiments, the compositions of this disclosure comprise a mixture of an anhydrous crystalline hemi-oxalate tianeptine salt and an anhydrous crystalline mono-oxalate tianeptine salt.

In some embodiments, the compositions of this disclosure comprise two or more layers of tianeptine oxalate, each layer comprising one or more of an anhydrous crystalline hemi-oxalate tianeptine salt, an anhydrous crystalline mono-oxalate tianeptine salt, a mixture thereof or polymorphs of any of them. In some embodiments, the compositions of this disclosure comprise two or more layers of tianeptine oxalate, each layer comprising one or more of an anhydrous crystalline hemi-oxalate tianeptine salt or a polymorph thereof. In some embodiments, the compositions of this disclosure comprise two or more layers of tianeptine oxalate, each layer comprising one or more of an anhydrous crystalline mono-oxalate tianeptine salt or a polymorph thereof. In some embodiments, the compositions of this disclosure comprise two or more layers of tianeptine oxalate, each layer comprising one or more of a mixture of an anhydrous crystalline hemi-oxalate tianeptine salt and an anhydrous crystalline mono-oxalate tianeptine salt or polymorphs thereof. In some embodiments, the compositions of this disclosure comprise two or more layers of tianeptine oxalate, each layer comprising one or more of an anhydrous crystalline hemi-oxalate tianeptine salt. In some embodiments, the compositions of this disclosure comprise two or more layers of tianeptine oxalate, each layer comprising one or more of an anhydrous crystalline mono-oxalate tianeptine salt. In some embodiments, the compositions of this disclosure comprise two or more layers of tianeptine oxalate, each layer comprising one or more of a mixture of an anhydrous crystalline hemi-oxalate tianeptine salt and an anhydrous crystalline mono-oxalate tianeptine salt.

In some embodiments, the compositions of this disclosure comprise a tianeptine oxalate, wherein the tianeptine oxalate is an anhydrous crystalline tianeptine hemi-oxalate, an anhydrous crystalline tianeptine mono-oxalate or a mixture thereof with a respective X-ray diffraction pattern (XRPD) as described in U.S. Pat. No. 10,449,203 (see, e.g., Figures 2, 9 and 10 and Tables 10 and 19).

In some embodiments, the compositions of this disclosure comprise a tianeptine oxalate, wherein the tianeptine oxalate is an anhydrous crystalline hemi-oxalate salt that exhibits an X-ray diffraction pattern (XPRD) comprising at least one peak selected from the group consisting of 8.2, 8.6, 9.1, and 9.5 degrees 2θ±0.3 degrees 2θ. In some embodiments, the compositions of this disclosure comprise a tianeptine oxalate, wherein the tianeptine oxalate is an anhydrous crystalline hemi-oxalate salt that exhibits an XRPD pattern comprising at least one additional peak selected from the group consisting of 4.5, 11.5, 14.2, 15.2, 15.8, 16.2, 19.2, 22.1, 23.9, 26.9, and 27.4 degrees 2θ 0.3 degrees 2θ. In some embodiments, the compositions of this disclosure comprise a tianeptine oxalate, wherein the tianeptine oxalate is an anhydrous crystalline hemi-oxalate salt that exhibits an XRPD pattern comprising at least one peak selected from the group consisting of 8.2, 8.6, 9.1, and 9.5 degrees 2θ±0.3 degrees 2θ and at least one additional peak selected from the group consisting of 4.5, 11.5, 14.2, 15.2, 15.8, 16.2, 19.2, 22.1, 23.9, 26.9, and 27.4 degrees 2θ±0.3 degrees 2θ.

In some embodiments, the compositions of this disclosure comprise a tianeptine oxalate, wherein the tianeptine oxalate is an anhydrous crystalline mono-oxalate salt that exhibits an X-ray diffraction pattern (XRPD) comprising at least one peak selected from the group consisting of 10.1 and 10.5 degrees 2θ±0.3 degrees 2θ. In some embodiments, the compositions of this disclosure comprise a tianeptine oxalate, wherein the tianeptine oxalate is an anhydrous crystalline mono-oxalate salt that exhibits an XRPD pattern comprising at least one additional peak selected from the group consisting of 7.5, 8.3, 11.9, 14.7, 16.2, 16.3, 17.9, 18.7, 21.0, 21.7, and 22.1 degrees 2θ±0.3 degrees 2θ. In some embodiments, the compositions of this disclosure comprise a tianeptine oxalate, wherein the tianeptine oxalate is an anhydrous crystalline mono-oxalate salt that exhibits an XRPD pattern comprising at least one peak selected from the group consisting of 10.1 and 10.5 degrees 2θ±0.3 degrees 2θ and at least one additional peak selected from the group consisting of 7.5, 8.3, 11.9, 14.7, 16.2, 16.3, 17.9, 18.7, 21.0, 21.7, and 22.1 degrees 2θ±0.3 degrees 2θ.

In some embodiments, the compositions of this disclosure comprise a tianeptine oxalate, wherein the tianeptine oxalate is a mixture of an anhydrous crystalline hemi-oxalate salt and an anhydrous crystalline mono-oxalate salt that exhibits an X-ray diffraction pattern (XRPD) comprising at least one peak selected from the group consist of 10.1 and 10.5 degrees 2θ±0.3 degrees 2θ. In some embodiments, the compositions of this disclosure comprise a tianeptine oxalate, wherein the tianeptine oxalate is a mixture of an anhydrous crystalline hemi-oxalate salt and an anhydrous crystalline mono-oxalate salt that exhibits an XRPD pattern further comprising at least one additional peak selected from the group consisting of 7.5, 8.3, 11.9, 14.7, 16.2, 16.3, 17.9, 18.7, 21.0, 21.7, and 22.1 degrees 2θ±0.3 degrees 2θ. In some embodiments, the compositions of this disclosure comprise a tianeptine oxalate, wherein the tianeptine oxalate is a mixture of an anhydrous crystalline hemi-oxalate salt and an anhydrous crystalline mono-oxalate salt that exhibits an XRPD pattern further comprising at least one peak selected from the group consist of 10.1 and 10.5 degrees 2θ±0.3 degrees 2θ and at least one additional peak selected from the group consisting of 7.5, 8.3, 11.9, 14.7, 16.2, 16.3, 17.9, 18.7, 21.0, 21.7, and 22.1 degrees 2θ±0.3 degrees 2θ.

In some embodiments, the compositions of this disclosure comprise naloxone or a pharmaceutically acceptable salt thereof. In some embodiments, the compositions of this disclosure comprise a naloxone acid salt. In some embodiments, the compositions of this disclosure comprise a naloxone hydrochloride. In some embodiments, the compositions of this disclosure comprise a naloxone hydrochloride dihydrate.

In some embodiments, the compositions of this disclosure comprise tianeptine, a pharmaceutically acceptable salt thereof or a polymorph of one or both that is present in an amount between 1% w/w to 30% w/w, between 5% w/w to 25% w/w, or between 10% w/w to 20% w/w. In some embodiments, the compositions of this disclosure comprise tianeptine, a pharmaceutically acceptable salt thereof or a polymorph of one or both that is present in an amount between 1% w/w to 30% w/w, between 5% w/w to 25% w/w, between 10% w/w to 20% w/w, between 1% w/w to 10% w/w, between 12% w/w to 18% w/w, or between 20% w/w to 30% w/w. In some embodiments, the compositions of this disclosure comprise tianeptine, a pharmaceutically acceptable salt thereof or a polymorph of one or both that is present in an amount between 1% w/w to 30% w/w. In some embodiments, the compositions of this disclosure comprise tianeptine, a pharmaceutically acceptable salt thereof or a polymorph of one or both that is present in an amount between 5% w/w to 25% w/w. In some embodiments, the compositions of this disclosure comprise tianeptine, a pharmaceutically acceptable salt thereof or a polymorph of one or both that is present in an amount between 10% w/w to 20% w/w. In some embodiments, the compositions of this disclosure comprise tianeptine, a pharmaceutically acceptable salt thereof or a polymorph of one or both that is present in an amount between 1% w/w to 10% w/w. In some embodiments, the compositions of this disclosure comprise tianeptine, a pharmaceutically acceptable salt thereof or a polymorph of one or both that is present in an amount between 12% w/w to 18% w/w. In some embodiments, the compositions of this disclosure comprise tianeptine, a pharmaceutically acceptable salt thereof or a polymorph of one or both that is present in an amount between 20% w/w to 30% w/w.

In some embodiments, the compositions of this disclosure comprise tianeptine, a pharmaceutically acceptable salt thereof or a polymorph of one or both that is present i in an amount equivalent to 14.28% w/w of the tianeptine free base. In some embodiments, the compositions of this disclosure comprise a pharmaceutically acceptable salt of tianeptine or a polymorph thereof that is present in an amount equivalent to 14.28% w/w of the tianeptine free base. In some embodiments, the compositions of this disclosure comprise a pharmaceutically acceptable salt of tianeptine that is present in an amount equivalent to 14.28% w/w of the tianeptine free base. In some embodiments, the compositions of this disclosure comprise tianeptine, a pharmaceutically acceptable salt thereof or a polymorph of one or both that is present in an amount equivalent to about 14.28% w/w of the tianeptine free base. In some embodiments, the compositions of this disclosure comprise a pharmaceutically acceptable salt of tianeptine or a polymorph thereof that is present in an amount equivalent to about 14.28% w/w of the tianeptine free base. In some embodiments, the compositions of this disclosure comprise a pharmaceutically acceptable salt of tianeptine that is present in an amount equivalent to about 14.28% w/w of the tianeptine free base.

In some embodiments, the compositions of this disclosure comprise a tianeptine hemi-oxalate salt that is present in the composition in an amount of 15.76% w/w. In some embodiments, the compositions of this disclosure comprise a tianeptine hemi-oxalate salt that is present in an amount of about 15.76% w/w.

In some embodiments, the compositions of this disclosure comprise naloxone or a pharmaceutically acceptable salt thereof that is present in an amount between 0.01% w/w to 5% w/w or between 0.1% w/w to 2% w/w. In some embodiments, the compositions of this disclosure comprise naloxone or a pharmaceutically acceptable salt thereof that is present in the composition in an amount between 0.01% w/w to 5% w/w, between 0.01% w/w to 1% w/w, between 0.1% w/w to 2% w/w, between 0.1% w/w to 1% w/w, or between 0.4% w/w to 4% w/w. In some embodiments, the compositions of this disclosure comprise naloxone or a pharmaceutically acceptable salt thereof that is present in an amount between 0.01% w/w to 5% w/w. In some embodiments, the compositions of this disclosure comprise naloxone or a pharmaceutically acceptable salt thereof that is present in an amount between 0.01% w/w to 1% w/w. In some embodiments, the compositions of this disclosure comprise naloxone or a pharmaceutically acceptable salt thereof that is present in an amount between 0.1% w/w to 2% w/w. In some embodiments, the compositions of this disclosure comprise naloxone or a pharmaceutically acceptable salt thereof that is present in an amount between 0.1% w/w to 1% w/w. In some embodiments, the compositions of this disclosure comprise naloxone or a pharmaceutically acceptable salt thereof that is present in an amount between 0.4% w/w to 4% w/w. In some embodiments, the compositions of this disclosure comprise naloxone or a pharmaceutically acceptable salt thereof that is present in an amount of less than 1% w/w or less than 0.5% w/w. In some embodiments, the compositions of this disclosure comprise naloxone or a pharmaceutically acceptable salt thereof that is present in an amount of less than 1% w/w. In some embodiments, the compositions of this disclosure comprise naloxone or a pharmaceutically acceptable salt thereof that is present in an amount of less than 0.5% w/w. In some embodiments, the compositions of this disclosure comprise naloxone or a pharmaceutically acceptable salt thereof that is present in an amount of less than 2% w/w. In some embodiments, the compositions of this disclosure comprise naloxone or a pharmaceutically acceptable salt thereof that is present in an amount of less than 3% w/w. In some embodiments, the compositions of this disclosure comprise naloxone or a pharmaceutically acceptable salt thereof that is present in an amount of less than 4% w/w. In some embodiments, the compositions of this disclosure comprise naloxone or a pharmaceutically acceptable salt thereof that is present in an amount of less than 5% w/w.

In some embodiments, the compositions of this disclosure comprise naloxone or a pharmaceutically acceptable salt thereof that is present in an amount equivalent to 0.40% w/w of the naloxone free base. In some embodiments, the compositions of this disclosure comprise a pharmaceutically acceptable salt of naloxone. In some embodiments, the compositions of this disclosure comprise a pharmaceutically acceptable salt of naloxone, wherein the pharmaceutically acceptable salt is naloxone hydrochloride. In some embodiments, the compositions of this disclosure comprise a pharmaceutically acceptable salt of naloxone, wherein the pharmaceutically acceptable salt is naloxone hydrochloride dihydrate. In some embodiments, the compositions of this disclosure comprise naloxone hydrochloride dihydrate that is present in an amount of 0.49% w/w. In some embodiments, the compositions of this disclosure comprise naloxone hydrochloride dihydrate that is present in an amount of about 0.49% w/w.

In some embodiments, the compositions of this disclosure comprise tianeptine, a pharmaceutically acceptable salt thereof or a polymorphs of one or both that is present in an amount between 0.1 mg to 60 mg, between 1 mg to 50 mg, or between 10 mg to 40 mg. In some embodiments, the compositions of this disclosure comprise tianeptine, a pharmaceutically acceptable salt thereof or a polymorph of one or both that is present in an amount between 0.1 mg to 60 mg, between 1 mg to 50 mg, between 10 mg to 40 mg, between 15 mg to 25 mg, or between 25 mg to 45 mg. In some embodiments, the compositions of this disclosure comprise tianeptine, a pharmaceutically acceptable salt thereof or a polymorph of one or both that is present in an amount between 0.1 mg to 60 mg. In some embodiments, the compositions of this disclosure comprise tianeptine, a pharmaceutically acceptable salt thereof or a polymorph of one or both that is present in an amount between 1 mg to 50 mg. In some embodiments, the compositions of this disclosure comprise tianeptine, a pharmaceutically acceptable salt thereof or a polymorph of one or both that is present in an amount between 10 mg to 40 mg. In some embodiments, the compositions of this disclosure comprise tianeptine, a pharmaceutically acceptable salt thereof or a polymorph of one or both that is present in an amount between 15 mg to 25 mg. In some embodiments, the compositions of this disclosure comprise tianeptine, a pharmaceutically acceptable salt thereof or a polymorph of one or both that is present in an amount between 25 mg to 45 mg. In some embodiments, the compositions of this disclosure comprise tianeptine, a pharmaceutically acceptable salt thereof or a polymorph of one or both that is present in an amount of 50 mg or less, 45 mg or less, or 40 mg or less. In some embodiments, the compositions of this disclosure comprise tianeptine, a pharmaceutically acceptable salt thereof or a polymorph of one or both that is present in an amount of 50 mg or less. In some embodiments, the compositions of this disclosure comprise tianeptine, a pharmaceutically acceptable salt thereof or a polymorph of one or both that is present in an amount of 45 mg or less. In some embodiments, the compositions of this disclosure comprise tianeptine, a pharmaceutically acceptable salt thereof or a polymorph of one or both that is present in an amount of 40 mg or less.

In some embodiments, the compositions of this disclosure comprise tianeptine, a pharmaceutically acceptable salt thereof or a polymorph of one or both that is present in an amount equivalent to 35.7 mg of the tianeptine free base. In some embodiments, the composition of this disclosure comprises a pharmaceutically acceptable salt of tianeptine or a polymorph thereof that is present in the composition in an amount equivalent to 35.7 mg of the tianeptine free base. In some embodiments, the composition of this disclosure comprises a pharmaceutically acceptable salt of tianeptine that is present in the composition in an amount equivalent to 35.7 mg of the tianeptine free base. In some embodiments, the composition of this disclosure comprises tianeptine, a pharmaceutically acceptable salt thereof or a polymorph of one or both that is present in an amount equivalent to about 35.7 mg of the tianeptine free base. In some embodiments, the compositions of this disclosure comprise a pharmaceutically acceptable salt of tianeptine or a polymorph thereof that is present in an amount equivalent to about 35.7 mg of the tianeptine free base. In some embodiments, the compositions of this disclosure comprise a pharmaceutically acceptable salt of tianeptine that is present in an amount equivalent to about 35.7 mg of the tianeptine free base.

In some embodiments, the compositions of this disclosure comprise a pharmaceutically acceptable salt that is an anhydrous crystalline hemi-oxalate salt or a polymorph thereof. In some embodiments, the compositions of this disclosure comprise an anhydrous crystalline tianeptine hemi-oxalate salt or polymorph thereof that is present in an amount of 39.4 mg. In some embodiments, the compositions of this disclosure comprise an anhydrous crystalline tianeptine hemi-oxalate salt that is present in the composition in an amount of about 39.4 mg.

In some embodiments, the compositions of this disclosure comprise naloxone or a pharmaceutically acceptable salt thereof that is present in an amount between 0.01 mg to 10 mg, between 0.1 mg to 5 mg, or between 0.5 mg to 2 mg. In some embodiments, the compositions of this disclosure comprise naloxone or a pharmaceutically acceptable salt thereof that is present in an amount between 0.01 mg to 10 mg, between 0.1 mg to 5 mg, between 0.5 mg to 2 mg, or between 1 mg to 5 mg. In some embodiments, the compositions of this disclosure comprise naloxone or a pharmaceutically acceptable salt thereof that is present in an amount of between 0.01 mg to 10 mg. In some embodiments, the compositions of this disclosure comprise naloxone or a pharmaceutically acceptable salt thereof that is present in an amount between 0.1 mg to 5 mg. In some embodiments, the compositions of this disclosure comprise naloxone or a pharmaceutically acceptable salt thereof that is present in an amount of between 0.5 mg to 2 mg. In some embodiments, the compositions of this disclosure comprise naloxone or a pharmaceutically acceptable salt thereof that is present in an amount of between 1 mg to 5 mg. In some embodiments, the compositions of this disclosure comprise naloxone or a pharmaceutically acceptable salt thereof that is present in an amount of 5 mg or less, or 2 mg or less. In some embodiments, the compositions of this disclosure comprise naloxone or a pharmaceutically acceptable salt thereof that is present in an amount of 5 mg or less. In some embodiments, the compositions of this disclosure comprise naloxone or a pharmaceutically acceptable salt thereof that is present in an amount of 2 mg or less.

In some embodiments, the compositions of this disclosure comprise naloxone or a pharmaceutically acceptable salt thereof that is present in an amount equivalent to 1 mg of the naloxone free base. In some embodiments, the compositions of this disclosure comprise a pharmaceutically acceptable salt of naloxone that is present in an amount equivalent to 1 mg of the naloxone free base. In some embodiments, the compositions of this disclosure comprise naloxone or a pharmaceutically acceptable salt thereof that is present in an amount equivalent to about 1 mg of the naloxone free base. In some embodiments, the compositions of this disclosure comprise a pharmaceutically acceptable salt of naloxone that is present in an amount equivalent to about 1 mg of the naloxone free base.

In some embodiments, the compositions of this disclosure comprise a pharmaceutically acceptable salt of naloxone. In some embodiments, the compositions of this disclosure comprise a pharmaceutically acceptable salt of naloxone, wherein the pharmaceutically acceptable salt is an acid salt. In some embodiments, the compositions of this disclosure comprise a pharmaceutically acceptable salt of naloxone, wherein the pharmaceutically acceptable salt is naloxone hydrochloride. In some embodiments, the compositions of this disclosure comprise a pharmaceutically acceptable salt of naloxone, wherein the pharmaceutically acceptable salt is naloxone hydrochloride dihydrate. In some embodiments, the compositions of this disclosure comprise naloxone hydrochloride dihydrate that is present in an amount of 1.22 mg. In some embodiments, the compositions of this disclosure comprise naloxone hydrochloride dihydrate that is present in an amount of about 1.22 mg.

In some embodiments, the compositions of this disclosure further comprise one or more excipients. In some embodiments, the compositions of this disclosure further comprise one or more of a filler, a controlled release polymer, a lubricant, a glidant, a pH adjusting agent, or a buffer. In some embodiments, the compositions of this disclosure further comprise one or more of a filler. In some embodiments, the compositions of this disclosure further comprise one or more of a controlled release polymer. In some embodiments, the compositions of this disclosure further comprise one or more of a lubricant. In some embodiments, the compositions of this disclosure further comprise one or more of a glidant. In some embodiments, the compositions of this disclosure further comprise one or more of a pH adjusting agent. In some embodiments, the compositions of this disclosure further comprise one or more of a buffer.

In some embodiments, the compositions of this disclosure further comprise a filler that is selected from the group consisting of microcrystalline cellulose, gelatin, sucrose, starch, acacia, tragacanth, alginic acid, cellulose, methyl cellulose, ethyl cellulose, hydroxypropyl methyl cellulose, hydroxypropyl cellulose, sodium carboxymethyl cellulose, polyvinyl pyrrolidone, polyethylene glycol, polyvinyl pyrrolidone, lactose monohydrate and cross-linked polyvinyl pyrrolidone. In some embodiments of the compositions of this disclosure, the filler is microcrystalline cellulose. In some embodiments of the compositions of this disclosure, the filler is gelatin. In some embodiments of the compositions of this disclosure, the filler is sucrose. In some embodiments of the compositions of this disclosure, the filler is starch. In some embodiments of the compositions of this disclosure, the filler is acacia. In some embodiments of the compositions of this disclosure, the filler is tragacanth. In some embodiments of the compositions of this disclosure, the filler is alginic acid. In some embodiments of the compositions of this disclosure, the filler is cellulose. In some embodiments of the compositions of this disclosure, the filler is methyl cellulose. In some embodiments of the compositions of this disclosure, the filler is ethyl cellulose. In some embodiments of the compositions of this disclosure, the filler is hydroxypropyl methyl cellulose. In some embodiments of the compositions of this disclosure, the filler is hydroxypropyl cellulose. In some embodiments of the compositions of this disclosure, the filler is sodium carboxymethyl cellulose. In some embodiments of the compositions of this disclosure, the filler is polyvinyl pyrrolidone. In some embodiments of the compositions of this disclosure, the filler is polyethylene glycol. In some embodiments of the compositions of this disclosure, the filler is polyvinyl pyrrolidone. In some embodiments of the compositions of this disclosure, the filler is lactose monohydrate. In some embodiments of the compositions of this disclosure, the filler is cross-linked polyvinyl pyrrolidone. In some embodiments of the compositions of this disclosure, the filler is present in an amount between 0.5% w/w to 50% w/w. In some embodiments of the compositions of this disclosure, the filler is present in an amount between 0.5% w/w to 15% w/w. In some embodiments of the compositions of this disclosure, the filler is present in an amount between 5% w/w to 15% w/w. In some embodiments of the compositions of this disclosure, the filler is present in an amount between 15% w/w to 30% w/w. In some embodiments of the compositions of this disclosure, the filler is present in an amount between 30% w/w to 50% w/w. In some embodiments of the compositions of this disclosure, the filler is present in an amount of 1.0% w/w. In some embodiments of the compositions of this disclosure, the filler is present in an amount of 7.5% w/w. In some embodiments of the compositions of this disclosure, the filler is present in an amount of 48.55% w/w. In some embodiments of the compositions of this disclosure, the filler is present in an amount of about 1.0% w/w. In some embodiments of the compositions of this disclosure, the filler is present in an amount of about 7.5% w/w. In some embodiments of the compositions of this disclosure, the filler is present in an amount of about 48.55% w/w.

In some embodiments, the compositions of this disclosure further comprise a controlled release polymer that is selected from the group consisting of hydroxypropyl methylcellulose, hydroxypropyl cellulose, polyvinyl pyrrolidone, carboxymethyl cellulose, methylcellulose, hydroxyethyl cellulose, ethyl hydroxyethyl cellulose, sodium carboxymethyl cellulose, sodium alginate, xanthan gum, carrageenan, chitosan, guar gum, pectin, polyethylene oxide, ethylcellulose, hypromellose acetate succinate, cellulose acetate and cellulose acetate propionate. In some embodiments of the compositions of this disclosure, the controlled release polymer is hydroxypropyl methylcellulose. In some embodiments of the compositions of this disclosure, the controlled release polymer is hydroxypropyl cellulose. In some embodiments of the compositions of this disclosure, the controlled release polymer is polyvinyl pyrrolidone. In some embodiments of the compositions of this disclosure, the controlled release polymer is carboxymethyl cellulose. In some embodiments of the compositions of this disclosure, the controlled release polymer is methylcellulose. In some embodiments of the compositions of this disclosure, the controlled release polymer is hydroxyethyl cellulose. In some embodiments of the compositions of this disclosure, the controlled release polymer is ethyl hydroxyethyl cellulose. In some embodiments of the compositions of this disclosure, the controlled release polymer is sodium carboxymethyl cellulose. In some embodiments of the compositions of this disclosure, the controlled release polymer is sodium alginate. In some embodiments of the compositions of this disclosure, the controlled release polymer is xanthan gum. In some embodiments of the compositions of this disclosure, the controlled release polymer is carrageenan. In some embodiments of the compositions of this disclosure, the controlled release polymer is chitosan. In some embodiments of the compositions of this disclosure, the controlled release polymer is guar gum. In some embodiments of the compositions of this disclosure, the controlled release polymer is pectin. In some embodiments of the compositions of this disclosure, the controlled release polymer is polyethylene oxide. In some embodiments of the compositions of this disclosure, the controlled release polymer is ethylcellulose. In some embodiments of the compositions of this disclosure, the controlled release polymer is hypromellose acetate succinate. In some embodiments of the compositions of this disclosure, the controlled release polymer is cellulose acetate. In some embodiments of the compositions of this disclosure, the controlled release polymer is cellulose acetate propionate. In some embodiments of the compositions of this disclosure, the controlled release polymer is present in the composition of this disclosure in an amount between 5% w/w to 30% w/w. In some embodiments of the compositions of this disclosure, the controlled release polymer is present in an amount between 5% w/w to 15% w/w. In some embodiments of the compositions of this disclosure, the controlled release polymer is present in an amount between 15% w/w to 30% w/w. In some embodiments of the compositions of this disclosure, the controlled release polymer is present in an amount of 7.5% w/w. In some embodiments of the compositions of this disclosure, the controlled release polymer is present in an amount of 25% w/w. In some embodiments of the compositions of this disclosure, the controlled release polymer is present in an amount of about 7.5% w/w. In some embodiments of the compositions of this disclosure, the controlled release polymer is present in an amount of about 25% w/w.

In some embodiments, the compositions of this disclosure further comprise a lubricant that is selected from the group consisting of magnesium stearate, calcium stearate, zinc stearate, stearic acid, myristic acid, palmitic acid, sodium stearyl fumarate, boric acid, carbowax (PEG) 4000/6000, sodium oleate, sodium benzoate, sodium acetate, sodium lauryl sulfate, magnesium lauryl sulfate, glyceride esters, glyceryl monostearate, glyceryl tribehenate, glyceryl dibehenate, sugar esters, sorbitan monostearate, sucrose monopalmitate, waxes, sterowet, glyceryl behenate, liquid paraffin and talc. In some embodiments of the compositions of this disclosure, the lubricant is magnesium stearate. In some embodiments of the compositions of this disclosure, the lubricant is calcium stearate. In some embodiments of the compositions of this disclosure, the lubricant is zinc stearate. In some embodiments of the compositions of this disclosure, the lubricant is stearic acid. In some embodiments of the compositions of this disclosure, the lubricant is myristic acid. In some embodiments of the compositions of this disclosure, the lubricant is palmitic acid. In some embodiments of the compositions of this disclosure, the lubricant is sodium stearyl fumarate. In some embodiments of the compositions of this disclosure, the lubricant is boric acid. In some embodiments of the compositions of this disclosure, the lubricant is carbowax (PEG) 4000/6000. In some embodiments of the compositions of this disclosure, the lubricant is sodium oleate. In some embodiments of the compositions of this disclosure, the lubricant is sodium benzoate. In some embodiments of the compositions of this disclosure, the lubricant is sodium acetate. In some embodiments of the compositions of this disclosure, the lubricant is sodium lauryl sulfate. In some embodiments of the compositions of this disclosure, the lubricant is magnesium lauryl sulfate. In some embodiments of the compositions of this disclosure, the lubricant is glyceride esters. In some embodiments of the compositions of this disclosure, the lubricant is glyceryl monostearate. In some embodiments of the compositions of this disclosure, the lubricant is glyceryl tribehenate. In some embodiments of the compositions of this disclosure, the lubricant is glyceryl dibehenate. In some embodiments of the compositions of this disclosure, the lubricant is sugar esters. In some embodiments of the compositions of this disclosure, the lubricant is sorbitan monostearate. In some embodiments of the compositions of this disclosure, the lubricant is sucrose monopalmitate. In some embodiments of the compositions of this disclosure, the lubricant is waxes. In some embodiments of the compositions of this disclosure, the lubricant is sterowet. In some embodiments of the compositions of this disclosure, the lubricant is glyceryl behenate. In some embodiments of the compositions of this disclosure, the lubricant is liquid paraffin. In some embodiments of the compositions of this disclosure, the lubricant is talc. In some embodiments of the compositions of this disclosure, the lubricant is present in an amount between 0.1% w/w to 5% w/w. In some embodiments of the compositions of this disclosure, the lubricant is present in an amount between 0.5% w/w to 2% w/w. In some embodiments of the compositions of this disclosure, the lubricant is present in an amount between 2% w/w to 5% w/w. In some embodiments of the compositions of this disclosure, the lubricant is present in an amount of 1.0% w/w. In some embodiments of the compositions of this disclosure, the lubricant is present in an amount of about 1.0% w/w.

In some embodiments, the compositions of this disclosure further comprise a glidant that is selected from the group consisting of starch, cornstarch, silica derivatives, fumed silica, colloidal silicon dioxide, hydrophilic fumed silica, syloid, pyrogenic silica, ascorbyl palmitate, calcium palmitate, magnesium stearate, talc and hydrated sodium sulfoaluminate. In some embodiments of the compositions of this disclosure, the glidant is starch. In some embodiments of the compositions of this disclosure, the glidant is cornstarch. In some embodiments of the compositions of this disclosure, the glidant is silica derivatives. In some embodiments of the compositions of this disclosure, the glidant is fumed silica. In some embodiments of the compositions of this disclosure, the glidant is colloidal silicon dioxide. In some embodiments, of the compositions of this disclosure the glidant is hydrophilic fumed silica. In some embodiments of the compositions of this disclosure, the glidant is syloid. In some embodiments of the compositions of this disclosure, the glidant is pyrogenic silica. In some embodiments of the compositions of this disclosure, the glidant is ascorbyl palmitate. In some embodiments of the compositions of this disclosure, the glidant is calcium palmitate. In some embodiments of the compositions of this disclosure, the glidant is magnesium stearate. In some embodiments of the compositions of this disclosure, the glidant is talc. In some embodiments of the compositions of this disclosure, the glidant is hydrated sodium sulfoaluminate. In some embodiments of the compositions of this disclosure, the glidant is present in an amount between 0.1% w/w to 5% w/w. In some embodiments of the compositions of this disclosure, the glidant is present in an amount between 0.1% w/w to 1% w/w. In some embodiments of the compositions of this disclosure, the glidant is present an amount between 1% w/w to 5% w/w. In some embodiments of the compositions of this disclosure, the glidant is present in an amount of 0.2% w/w. In some embodiments of the compositions of this disclosure, the glidant is present in an amount of about 0.2% w/w.

In some embodiments, the compositions of this disclosure further comprise a pH adjusting agent that is selected from the group consisting of citric acid monohydrate, trisodium citrate dihydrate, sodium acetate, acetic acid, tartaric acid, sodium hydrogen carbonate, potassium citrate monohydrate, sodium citrate, tromethamine (Tris), sodium hydroxide, sodium phosphate, disodium hydrogen phosphate, sodium bisphosphate, boric acid, sodium borate, benzoic acid, sodium benzoate, aspartic acid, and maleic acid. In some embodiments of the compositions of this disclosure, the pH adjusting agent is citric acid monohydrate. In some embodiments of the compositions of this disclosure, the pH adjusting agent is trisodium citrate dihydrate. In some embodiments of the compositions of this disclosure, the pH adjusting agent is sodium acetate. In some embodiments of the compositions of this disclosure, the pH adjusting agent is acetic acid. In some embodiments of the compositions of this disclosure, the pH adjusting agent is tartaric acid. In some embodiments of the compositions of this disclosure, the pH adjusting agent is sodium hydrogen carbonate. In some embodiments of the compositions of this disclosure, the pH adjusting agent is potassium citrate monohydrate. In some embodiments of the compositions of this disclosure, the pH adjusting agent is sodium citrate. In some embodiments of the compositions of this disclosure, the pH adjusting agent is tromethamine (Tris). In some embodiments of the compositions of this disclosure, the pH adjusting agent is sodium hydroxide. In some embodiments of the compositions of this disclosure, the pH adjusting agent is sodium phosphate. In some embodiments of the compositions of this disclosure, the pH adjusting agent is disodium hydrogen phosphate. In some embodiments of the compositions of this disclosure, the pH adjusting agent is sodium bisphosphate. In some embodiments of the compositions of this disclosure, the pH adjusting agent is boric acid. In some embodiments of the compositions of this disclosure, the pH adjusting agent is sodium borate. In some embodiments of the compositions of this disclosure, the pH adjusting agent is benzoic acid. In some embodiments of the compositions of this disclosure, the pH adjusting agent is sodium benzoate. In some embodiments of the compositions of this disclosure, the pH adjusting agent is aspartic acid. In some embodiments of the compositions of this disclosure, the pH adjusting agent is maleic acid. In some embodiments of the compositions of this disclosure, the pH adjusting agent is present in an amount between 0.1% to 2% w/w. In some embodiments of the compositions of this disclosure, the pH adjusting agent is present in an amount between 0.1% to 1% w/w. In some embodiments of the compositions of this disclosure, the pH adjusting agent is present in an amount between 0.5% to 1.5% w/w. In some embodiments of the compositions of this disclosure, the pH adjusting agent is present in an amount between 1% to 2% w/w. In some embodiments of the compositions of this disclosure, the pH adjusting agent is present in an amount of 0.5% w/w. In some embodiments of the compositions of this disclosure, the pH adjusting agent is present in an amount of about 0.5% w/w.

In some embodiments, the compositions of this disclosure further comprise a buffer that is selected from the group consisting of trisodium citrate dihydrate, citric acid, boric acid, sodium citrate, potassium bitartrate, calcium acetate, ammonium phosphate, calcium chloride, citric acid salts, lactic acid, magnesium trisilicate, phosphoric acid, potassium, potassium citrate, potassium phosphate, sodium acetate, sodium borate, sodium lactate, succinic acid, and monothioglycerol. In some embodiments of the compositions of this disclosure, the buffer is trisodium citrate dihydrate. In some embodiments of the compositions of this disclosure, the buffer is citric acid. In some embodiments of the compositions of this disclosure, the buffer is boric acid. In some embodiments of the compositions of this disclosure, the buffer is sodium citrate. In some embodiments, the buffer is potassium bitartrate. In some embodiments of the compositions of this disclosure, the buffer is calcium acetate. In some embodiments of the compositions of this disclosure, the buffer is ammonium phosphate. In some embodiments of the compositions of this disclosure, the buffer is calcium chloride. In some embodiments of the compositions of this disclosure, the buffer is citric acid salts. In some embodiments of the compositions of this disclosure, the buffer is lactic acid. In some embodiments of the compositions of this disclosure, the buffer is magnesium trisilicate. In some embodiments of the compositions of this disclosure, the buffer is phosphoric acid. In some embodiments of the compositions of this disclosure, the buffer is potassium. In some embodiments of the compositions of this disclosure, the buffer is potassium citrate. In some embodiments of the compositions of this disclosure, the buffer is potassium phosphate. In some embodiments of the compositions of this disclosure, the buffer is sodium acetate. In some embodiments of the compositions of this disclosure, the buffer is sodium borate. In some embodiments of the compositions of this disclosure, the buffer is sodium lactate. In some embodiments of the compositions of this disclosure, the buffer is succinic acid. In some embodiments of the compositions of this disclosure, the buffer is monothioglycerol. In some embodiments of the compositions of this disclosure, the buffer is present in an amount between 0.1% w/w to 2% w/w. In some embodiments of the compositions of this disclosure, the buffer is present in an amount between 0.1% w/w to 1% w/w. In some embodiments of the compositions of this disclosure, the buffer is present in an amount between 0.5% w/w to 1.5% w/w. In some embodiments of the compositions of this disclosure, the buffer is present in an amount between 1% w/w to 2% w/w. In some embodiments of the compositions of this disclosure, the buffer is present in an amount of 0.5% w/w. In some embodiments of the compositions of this disclosure, the buffer is present in an amount of about 0.5% w/w.

In some embodiments, the compositions of this disclosure further comprise a pharmaceutically acceptable carrier.

In some embodiments, the compositions of this disclosure are in the form of a tablet, a mini-tablet, a chewable tablet, an orodispersible tablet, dissolvable tablet, a scored tablet, a coated tablet, a suppository, or a granule. In some embodiments, the compositions of this disclosure are in the form of a tablet. In some embodiments, the compositions of this disclosure are in the form of a mini-tablet. In some embodiments, the compositions of this disclosure are in the form of a chewable tablet. In some embodiments, the compositions of this disclosure are in the form of an orodispersible tablet. In some embodiments, the compositions of this disclosure are in the form of a dissolvable tablet. In some embodiments, the compositions of this disclosure are in the form of a scored tablet. In some embodiments, the compositions of this disclosure are in the form of a coated tablet. In some embodiments, the compositions of this disclosure are in the form of a suppository. In some embodiments, the compositions of this disclosure are in the form of a granule.

In some embodiments, the compositions of this disclosure are in the form of a tablet that is formulated so as to be capable of immediate release, controlled release, sustained release, extended release, or slow release of one or both of tianeptine, a pharmaceutically acceptable salt thereof or a polymorph of one or both and naloxone or a pharmaceutically salt thereof. In some embodiments, the compositions of this disclosure are in the form of a tablet that is formulated so as to be capable of immediate release of one or both of tianeptine, a pharmaceutically acceptable salt thereof or a polymorph of one or both and naloxone or a pharmaceutically salt thereof. In some embodiments, the compositions of this disclosure are in the form of a tablet that is formulated so as to be capable of controlled release of one or both of tianeptine, a pharmaceutically acceptable salt thereof or a polymorph of one or both and naloxone or a pharmaceutically salt thereof. In some embodiments, the compositions of this disclosure are in the form of a tablet that is formulated so as to be capable of sustained release of one or both of tianeptine, a pharmaceutically acceptable salt thereof or a polymorph of one or both and naloxone or a pharmaceutically salt thereof. In some embodiments, the compositions of this disclosure are in the form of a tablet that is formulated so as to be capable of extended release of one or both of tianeptine, a pharmaceutically acceptable salt thereof or a polymorph of one or both and naloxone or a pharmaceutically salt thereof. In some embodiments, the compositions of this disclosure are in the form of a tablet that is formulated so as to be capable of slow release of one or both of tianeptine, a pharmaceutically acceptable salt thereof or a polymorph of one or both and naloxone or a pharmaceutically salt thereof.

In some embodiments, the compositions of this disclosure are in the form of a tablet that is formulated so as to be capable of controlled release of tianeptine oxalate or a polymorph thereof and naloxone hydrochloride. In some embodiments, the compositions of this disclosure are in the form of a tablet that is formulated so as to be capable of controlled release of an anhydrous crystalline hemi-oxalate salt of tianeptine oxalate and naloxone hydrochloride dihydrate.

In some embodiments, the compositions of this disclosure are a controlled release formulation. In some embodiments, the compositions of this disclosure are a controlled release tablet formulation. In some embodiments, the compositions of this disclosure are a controlled release tablet formulation that is administered orally. In some embodiments, the compositions of this disclosure are a controlled release formulation that comprises tianeptine, a pharmaceutically acceptable salt thereof, or a polymorph of one or both and naloxone or a pharmaceutically acceptable sale thereof. In some embodiments, the compositions of this disclosure are a controlled release formulation that comprise tianeptine oxalate and naloxone hydrochloride. In some embodiments, the compositions of this disclosure are a controlled release formulation that comprise tianeptine hemi-oxalate salt and naloxone hydrochloride dihydrate. In some embodiments, the compositions of this disclosure are a controlled release formulation that comprise anhydrous crystalline tianeptine hemi-oxalate salt and naloxone hydrochloride dihydrate.

In some embodiments of the compositions of this disclosure, the tablet is characterized by a hardness of greater than 10 Kp. In some embodiments of the compositions of this disclosure, the tablet is characterized by a tensile strength of greater than 2 MPa.

Method of Preventing and/or Treating

In some aspects, the present disclosure provides methods of preventing and/or treating major depressive disorder (MDD) or one or more symptoms associated therewith, comprising administering to a subject in need thereof or at risk thereof, a composition of this disclosure. In some aspects, the present disclosure provides methods of preventing and/or treating major depressive disorder (MDD) or one or more symptoms associated therewith, comprising administering to a subject in need thereof or at risk thereof, a composition of this disclosure comprising tianeptine or a pharmaceutically acceptable salt thereof or polymorphs of one or both, and naloxone or a pharmaceutically acceptable salt thereof. In some embodiments of the methods of this disclosure, the tianeptine is a tianeptine oxalate salt. In some embodiments of the methods of this disclosure, the tianeptine oxalate is a tianeptine hemi-oxalate salt. In some embodiments of the methods of this disclosure, the tianeptine oxalate is a tianeptine mono-oxalate salt. In some embodiments of the methods of this disclosure, the tianeptine oxalate is a mixture of a tianeptine hemi-oxalate salt and a tianeptine mono-oxalate salt. In some embodiments, the method of this disclosure comprises administering a tianeptine hemi-oxalate salt, a tianeptine mono-oxalate salt or a mixture of them, and naloxone or a pharmaceutically acceptable salt thereof. In some embodiments, the method of this disclosure comprises administering a tianeptine hemi-oxalate salt and naloxone or a pharmaceutically acceptable salt thereof. In some embodiments, the method of this disclosure comprises administering a tianeptine mono-oxalate salt and naloxone or a pharmaceutically acceptable salt thereof. In some embodiments, the method of this disclosure comprises administering a mixture of a tianeptine hemi-oxalate salt and a tianeptine mono-oxalate salt, and naloxone or a pharmaceutically acceptable salt thereof.

In some embodiments, the methods of this disclosure comprise administering a composition of this disclosure one or more times daily to prevent and/or treat major depressive disorder (MDD) or one or more symptoms associated therewith in a subject in need thereof or at risk thereof. In some embodiments, the methods of this disclosure comprise administering a composition of this disclosure once daily to prevent and/or treat major depressive disorder (MDD) or one or more symptoms associated therewith in a subject in need thereof or at risk thereof. In some embodiments, the methods of this disclosure comprise administering a composition of this disclosure multiple times daily to prevent and/or treat major depressive disorder (MDD) or one or more symptoms associated therewith in a subject in need thereof or at risk thereof. In some embodiments, the methods of this disclosure comprise administering a composition of this disclosure at bedtime to prevent and/or treat major depressive disorder (MDD) or one or more symptoms associated therewith in a subject in need thereof or at risk thereof.

In some embodiments, the method of preventing and/or treating major depressive disorder (MDD) or one or more symptoms associated therewith in a subject in need thereof or at risk thereof comprises administering a composition of this disclosure orally, sublingually, buccally, palatially, rectally, or vaginally. In some embodiments, the method of preventing and/or treating major depressive disorder (MDD) or one or more symptoms associated therewith in a subject in need thereof or at risk thereof comprises administering a composition of this disclosure orally. In some embodiments, the method of preventing and/or treating major depressive disorder (MDD) or symptoms associated therewith in a subject in need thereof or at risk thereof comprises administering a composition of this disclosure sublingually. In some embodiments, the method of preventing and/or treating major depressive disorder (MDD) or one or more symptoms associated therewith in a subject in need thereof or at risk thereof comprises administering a composition of this disclosure buccally. In some embodiments, the method of preventing and/or treating major depressive disorder (MDD) or one or more symptoms associated therewith in a subject in need thereof or at risk thereof comprises administering a composition of this disclosure palatially. In some embodiments, the method of preventing and/or treating major depressive disorder (MDD) or one or more symptoms associated therewith in a subject in need thereof or at risk thereof comprises administering a composition of this disclosure rectally. In some embodiments, the method of preventing and/or treating major depressive disorder (MDD) or one or more symptoms associated therewith in a subject in need thereof or at risk thereof comprises administering a composition of this disclosure vaginally.

### Method of Manufacturing

In some aspects, the present disclosure provides a method of manufacturing compositions of this disclosure, wherein tianeptine, a pharmaceutically acceptable salt thereof or polymorph of one or both, naloxone or a pharmaceutically acceptable salt thereof, and other components of the compositions of this disclosure are combined. The skilled worker will understand the order of the combination and the amounts of each component given their knowledge in the art in view of the components and amounts of the various embodiments described herein. In some embodiments of the methods of manufacturing of this disclosure, the naloxone or a pharmaceutically acceptable salt thereof is mixed with a pH adjusting agent prior to its mixing with tianeptine, a pharmaceutically acceptable salt thereof or a polymorph of one or both, and with one or more excipients and other components of the composition of this disclosure. In some embodiments of the methods of manufacturing of this disclosure, naloxone is first mixed or combined with a pH adjusting agent. In some embodiments of the methods of manufacturing of this disclosure, naloxone or a pharmaceutically acceptable salt thereof is first mixed or combined with citric acid, trisodium citrate, or a combination thereof. In some embodiments of the methods of manufacturing of this disclosure, naloxone hydrochloride is first mixed or combined with citric acid, trisodium citrate, or a combination thereof. In some embodiments of the methods of manufacturing of this disclosure, naloxone hydrochloride dihydrate is first mixed or combined with citric acid, trisodium citrate, or a combination thereof. In some embodiments of the methods of manufacturing of this disclosure, naloxone hydrochloride dihydrate is first mixed or combined with citric acid, trisodium citrate, or a combination thereof prior to its mixing with tianeptine oxalate. In some embodiments of the methods of manufacturing of this disclosure, naloxone hydrochloride dihydrate is first mixed or combined with citric acid, trisodium citrate, or a combination thereof prior to its mixing with anhydrous crystalline tianeptine hemi-oxalate salt.

In some embodiments, the methods of manufacturing a composition of this disclosure, are characterized by naloxone or a pharmaceutically acceptable salt thereof that before mixing is comprised of particles that are less than 20 μm. In some embodiments of the methods of manufacturing of this disclosure, the addition of the pH adjusting agents to naloxone is by titration type blending and/or coating. In some embodiments of the methods of manufacturing of this disclosure, the addition of the one or more pH adjusting agents to naloxone is by titration type blending and/or coating. In some embodiments of the methods of manufacturing of this disclosure, a fine grade of naloxone is used where 90% of the naloxone particles are less than 20 μm, i.e., providing a uniform naloxone content. Without wishing to be bound by theory, the use of a fine grade of naloxone is believed to increase surface area and lead to enhanced degradation of naloxone. Without wishing to be bound by theory, it is also believed that to increase the stability of naloxone, pH adjusting agents (e.g., citric acid and trisodium citrate) can be mixed with naloxone.

In some embodiments of the methods of manufacturing the compositions of this disclosure comprises compression of the composition to form a tablet. In some embodiments of the methods of manufacturing of this disclosure, the methods do not affect the dissolution or release profile. In some embodiments of the methods of manufacturing of this disclosure, the compression of the composition to form a tablet does not affect the dissolution or release profile of tianeptine, pharmaceutically acceptable salts thereof or polymorphs of one or both and naloxone and pharmaceutically acceptable salts thereof. In some embodiments of the methods of manufacturing of this disclosure, the tablet of this disclosure is compressed to a hardness of greater than 10 Kp. In some embodiments of the methods of manufacturing of this disclosure, the tablet of this disclosure is compressed to a tensile strength of greater than 2 MPa.

In some embodiments, the methods and compositions of this disclosure prevent or reduce the potential for abuse of tianeptine, pharmaceutically acceptable salts thereof or polymorphs of one or both of them. In some embodiments, the methods and compositions of this disclosure prevent or reduce the potential for abuse of a pharmaceutically acceptable salt of tianeptine. In some embodiments, the compositions of this disclosure prevent or reduce the potential for abuse of tianeptine, pharmaceutically acceptable salts thereof or polymorphs of one or both of them. In some embodiments, the compositions of this disclosure prevent or reduce the potential for abuse of a pharmaceutically acceptable salt of tianeptine. In some embodiments, emulsification of the composition of this disclosure hinders or prevents separation of tianeptine, pharmaceutically acceptable salts thereof or polymorphs of one or both from naloxone. Emulsification occurs due to the presence of an effective amount of a lubricant (e.g., magnesium stearate) in some embodiments of the compositions of this disclosure. In some embodiments, a lubricant (e.g., magnesium stearate) of the composition hinders or prevents separation of tianeptine, pharmaceutically acceptable salts thereof or polymorphs thereof from naloxone.

### EXAMPLES

### Example 1. Pre-Clinical Dose Range Finding and Toxicity Studies in Rats

A 7-day dose range study to assess toxicity and toxicokinetic characteristics of tianeptine hemi-oxalate is performed in rats. Dosing formulations are prepared under yellow lighting and adjusted for pH at appropriate concentrations to meet dose level requirements. The control formulation (vehicle) is 20% Captisol in deionized water with a pH of about 7.6-7.8. The oxalate is added gradually into the vehicle to achieve a suspension at a target concentration and volume with a final pH between about 7.6-7.8. The dose level and experimental groups are described in Table 1 and Table 2. The formulation of the oxalate is administered to rats by oral gavage once per day for up to 7 days. The dose volume is based on the most recent body weight measurement of the rats. The values in ( ) are the equivalent salt dose levels/concentrations of the free base (tianeptine).

Blood samples are collected from Groups 5-8 through the sublingual vein (or other suitable vein) and are processed for plasma to analyze tianeptine hemi-oxalate for toxicokinetic characteristics. The samples are collected on Day 1 at post-dose collection intervals of 0.5, 1, 2, 4, 8 and 24 hours and on Day 7 at 0 hour (pre-dose) and post-dose collection intervals of 0.5, 1, 2, 4, 8 and 24 hours. Anon-compartmental approach consistent with the oral route of administration is used for parameter estimation including assessing one or more of tₘₐₓ, Cₘₐₓ, Cₘₐₓ/Dose, AUCₜₗₐₛₜ, AUCₜₗₐₛₜ/Dose, AUC₍₀₋ₜ₎, AUC₍₀₋ₜ₎/Dose, tₗₐₛₜ, and R_{AUC}. All parameters are generated from the hemi-oxalate composition concentrations in plasma from Days 1 and 7 whenever practical.

A 56-day study with 28-day recovery period to assess toxicity and toxicokinetic characteristics of the tianeptine hemi-oxalate and naloxone HCl dihydrate is performed in rats. Dosing formulations are prepared under yellow lighting and adjusted for pH at appropriate concentrations to meet dose level requirements. The control formulation (vehicle) is 20% Captisol in deionized water with a pH between about 7.6-7.8. The hemi-oxalate or the hemi oxalate in combination with the naloxone HCl is added gradually into the vehicle to achieve a suspension at a target concentration and volume with a final pH between about 7.6-7.8. The dose level and experimental groups are described in Table 3 and Table 4. The compositions (e.g., the hemi-oxalate with or without Naloxone) or vehicle control are administered to rats by oral gavage once per day for up to 56 days. The dose volume is based on the most recent body weight measurement of the rats. The values in ( ) are the equivalent salt dose levels/concentrations of the free base (tianeptine).

TABLE 1
7-day experimental design to assess clinical pathology

| | | | | | Main Study | |
|---|---|---|---|---|---|---|
| | | Dose | | Dose | Number | Number |
| | | Level | Dose | Concen- | of | of |
| | Com- | (mg/ | Volume | tration | Male | Female |
| Group | pound | kg/day) | (mL/kg) | (mg/mL) | Rats | Rats |
| 1 | Tianeptine | 0 | 10 | 0 | 5 | 5 |
| | Hemi- | | | | | |
| | oxalate | | | | | |
| 2* | Tianeptine | 180 (198) | 10 | 18 (19.8) | 5 | 5 |
| | Hemi- | | | | | |
| | oxalate | | | | | |
| 3 | Tianeptine | 30 (33) | 10 | 3 (3.3) | 5 | 5 |
| | Hemi- | | | | | |
| | oxalate | | | | | |
| 4 | Tianeptine | 90 (99) | 10 | 9 (9.9) | 5 | 5 |
| | Hemi- | | | | | |
| | oxalate | | | | | |

*Due to a lack of clinical observations of Group 4 at 90 mg/kg/day, Group 2 receives a dosing holiday and start dosing at 180 mg/kg/day starting on Day 10.

TABLE 2
7-day experimental design to assess toxicokinetic characteristics

| | | Dose | | Dose | Number | Number |
|---|---|---|---|---|---|---|
| | | Level | Dose | Concen- | of | of |
| | Com- | (mg/ | Volume | tration | Male | Female |
| Group | pound | kg/day) | (mL/kg) | (mg/mL) | Rats | Rats |
| 5 | Tianeptine | 10 (11) | 10 | 1 (1.1) | 6 | 6 |
| | Hemi- | | | | | |
| | oxalate | | | | | |
| 6 | Tianeptine | 30 (33) | 10 | 3 (3.3) | 6 | 6 |
| | Hemi- | | | | | |
| | oxalate | | | | | |
| 7 | Tianeptine | 90 (99) | 10 | 9 (9.9) | 6 | 6 |
| | Hemi- | | | | | |
| | oxalate | | | | | |
| 8** | Tianeptine | 180 (198) | 10 | 18 (19.8) | 4 | 4 |
| | Hemi- | | | | | |
| | oxalate | | | | | |

**Due to a lack of clinical observations of Group 4 at 90 mg/kg/day, additional animals not selected for study are assigned to Group 8. A full toxicokinetic profile are collected from these animals at the new dose level of 180 mg/kg/day.

TABLE 3
56-day experimental design to assess clinical pathology

| | | | | | Main Study | | Recovery Study | |
|---|---|---|---|---|---|---|---|---|
| | | Dose | | Dose | Number | Number | Number | Number |
| | | Level | Dose | Concen- | of | of | of | of |
| | | (mg/kg/ | Volume | tration | Male | Female | Male | Female |
| Group | Compound | day) | (mL/kg) | (mg/mL) | Rats | Rats | Rats | Rats |
| 1 | Vehicle | 0 | 10 | 0 | 10 | 10 | 6 | 6 |
| | Control | | | | | | | |
| 2 | Tianeptine | 10 (11) | 10 | 1 (1.1) | 10 | 10 | 0 | 0 |
| | Hemi- | | | | | | | |
| | oxalate | | | | | | | |
| 3 | Tianeptine | 30 (33) | 10 | 3 (3.3) | 10 | 10 | 0 | 0 |
| | Hemi- | | | | | | | |
| | oxalate | | | | | | | |
| 4 | Tianeptine | 90 (99) | 10 | 9 (9.9) | 10 | 10 | 6 | 6 |
| | Hemi- | | | | | | | |
| | oxalate | | | | | | | |
| 5 | Tianeptine | 90 | 10 | 9 | 10 | 10 | 6 | 6 |
| | Hemi- | (99) + | | (9.9) + | | | | |
| | oxalate + | 2.28 | | 0.228 | | | | |
| | Naloxone | (2.78) | | (0.278) | | | | |
| | HCl | | | | | | | |
| | dihydrate | | | | | | | |

TABLE 4
56-day experimental design to assess toxicokinetic characteristics

| | | Dose | | Dose | Number | Number |
|---|---|---|---|---|---|---|
| | | Level | Dose | Concen- | of | of |
| | Com- | (mg/ | Volume | tration | Male | Female |
| Group | pound | kg/day) | (mL/kg) | (mg/mL) | Rats | Rats |
| 6 | Vehicle | 0 | 10 | 0 | 3 | 3 |
| | Control | | | | | |
| 7 | Tianeptine | 10 (11) | 10 | 1 (1.1) | 6 | 6 |
| | Hemi- | | | | | |
| | oxalate | | | | | |
| 8 | Tianeptine | 30 (33) | 10 | 3 (3.3) | 6 | 6 |
| | Hemi- | | | | | |
| | oxalate | | | | | |
| 9 | Tianeptine | 90 (99) | 10 | 9 (9.9) | 6 | 6 |
| | Hemi- | | | | | |
| | oxalate | | | | | |
| 10 | Tianeptine | 90 | 10 | 9 | 6 | 6 |
| | Hemi- | (99) + | | (9.9) + | | |
| | oxalate + | | | | | |
| | Naloxone | 2.28 | | 0.228 | | |
| | HCl | (2.78) | | (0.278) | | |
| | dihydrate | | | | | |

Blood samples are collected from Groups 6-10 through the sublingual vein (or other suitable vein) and processed for plasma to analyze the hemi-oxalate and the naloxone for toxicokinetic characteristics. The samples are collected on Day 1 at post-dose collection intervals of 0.5, 1, 2, 4, 8 and 24 hours and on Day 56 at 0 hours (pre-dose) and post-dose collection intervals of 0.5, 1, 2, 4, 8 and 24 hours. A non-compartmental approach consistent with the oral route of administration is used for parameter estimation including assessing one or more of tₘₐₓ, Cₘₐₓ, Cₘₐₓ/Dose, AUCₜₗₐₛₜ, AUCₜₗₐₛₜ/Dose, AUC₍₀₋ₜ₎, AUC₍₀₋ₜ₎/Dose, tₗₐₛₜ, and R_{AUC}. All parameters are generated from hemi-oxalate and hemi-oxalate-Naloxone (Group 10 only) composition concentrations in plasma from Days 1 and 56 whenever practical.

Blood and urine samples are collected to assess clinical pathology (i.e., hematology, coagulation, clinical chemistry and urinalysis) in both the 7-day (Groups 1-4) and 56-day (Groups 1-5) rat studies. Blood samples are collected from the vena cava after carbon dioxide inhalation. The order of blooding is alternating (1 animal per sex from each dose group, then repeating) to reduce handling and time biases. Urine samples are collected from the animals for a period of at least 12 hours. The hematology parameters include one or more of leukocyte count (total and absolute differential), erythrocyte count, hemoglobin, hematocrit, absolute reticulocytes, mean corpuscular hemoglobin (calculated), mean corpuscular volume, mean corpuscular hemoglobin concentration (calculated), red cell distribution width (RDW), platelet count, and blood smear (preserve and stain). The coagulation parameters include one or more of prothrombin time, activated partial thromboplastin time, and fibrinogen. The clinical chemistry parameters include one or more of alkaline phosphatase, total bilirubin (with direct bilirubin if total bilirubin exceeds 1 mg/dL), aspartate aminotransferase, alanine aminotransferase, urea nitrogen, creatinine, total protein, albumin, globulin and A/G (albumin/globulin) ratio (calculated), glucose, total cholesterol, triglycerides, electrolytes (sodium, potassium, chloride), calcium, phosphorus, and sample quality. The urinalysis parameters include one or more of volume, color and appearance, specific gravity, pH, protein, glucose, bilirubin, ketones, occult blood, and urobilinogen.

Tissues are collected, weighed and preserved in 10% neutral buffered formalin from rats in the 7-day and 56-day Main Studies for histology processing and evaluation (e.g., animal ID, macroscopic evaluation (e.g., abnormalities), and/or microscopic evaluation). Tissues that are collected include one or more of artery (e.g., aorta), body cavity (e.g., nasal), bone marrow smear, bone marrow (e.g., sternum), bone (e.g., femur and/or sternum), brain, epididymis(ides), esophagus, eye(s), ganglion (e.g., dorsal root and/or lumbar), gland(s) (e.g., adrenal, clitoral, Harderian, lacrimal, mammary, parathyroid, pituitary, preputial, prostate, salivary (e.g., parotid, sublingual and/or mandibular), seminal vesicle, thyroid, and/or Zymbal's), gut-associated lymphoid tissue, heart, joint (e.g., femorotibial), kidney(s), large intestine (e.g., cecum, colon and/or rectum), larynx, liver, lung(s), lymph node(s) (e.g., mandibular and/or mesenteric), muscle (e.g., skeletal), nerve (e.g., optic, sciatic and/or tibial), ovary(ies), oviduct(s), pancreas, skin, small intestine (e.g., duodenum, ileum, and/or jejunum), spinal cord (e.g., cervical, thoracic, and/or lumbar), spleen, stomach, testis(es), thymus, tongue, trachea, ureter, urinary bladder, uterus, cervix, and vagina. Bone marrow smears are collected from the femur at scheduled and unscheduled necropsies for possible examination. Smears are not collected from animals that are found dead or from animals that are euthanized moribund and then stored in the refrigerator prior to necropsy. Bone marrow smears are allowed to air dry and are not fixed in formalin. Eyes and optic nerves are preserved in Davidson's fixative. Testes and epididymides are preserved in modified Davidson's fixative.

### Example 2. Pre-Clinical Toxicity Studies in Dogs

A two-phase study is performed with beagle dogs to (1) estimate the single-dose oral maximum tolerated dose (MTD) of tianeptine hemi-oxalate (Phase A) and (2) evaluate the toxicity and toxicokinetic of tianeptine hemi-oxalate when it is administered once daily via oral gavage for 7 consecutive days (Phase B). Dosing formulations are prepared under yellow lighting and are adjusted for pH at appropriate concentrations to meet dose level requirements. The control formulation (vehicle) is 20% Captisol in deionized water with a pH between about 7.6-7.8. Tianeptine hemi-oxalate is added gradually into the vehicle to achieve a suspension at a target concentration and volume with a final pH between about 7.6-7.8. The dose level and experimental groups are described in Table 5 and Table 6. The dose volume is based on the most recent body weight measurement. After each dose, and prior to removal of the gavage tube, the tube will be flushed with 10 mL of tap water. The values in ( ) are the equivalent salt dose levels/concentrations of the free base (tianeptine).

Blood and urine samples are collected twice during Pretest and once at Day 8 to assess clinical pathology (i.e., hematology, coagulation, clinical chemistry and urinalysis) of Groups 2-5. Blood samples are collected from the jugular vein (or other suitable vein). The order of bleeding is alternating (1 animal per sex from each dose group, then repeating) to reduce handling and time biases, unless concurrent with other timed functions. Urine samples are collected using steel pans placed under the cages for at least 16 hours. The hematology parameters include one or more of leukocyte count (total and absolute differential), erythrocyte count, hemoglobin, hematocrit, absolute reticulocytes, mean corpuscular hemoglobin (calculated), mean corpuscular volume, mean corpuscular hemoglobin concentration (calculated), red cell distribution width (RDW), platelet count, and blood smear (preserve and stain). The coagulation parameters include one or more of prothrombin time, activated partial thromboplastin, and fibrinogen. The clinical chemistry parameters include one or more of alkaline phosphatase, total bilirubin (with direct bilirubin if total bilirubin exceeds 1 mg/dL), aspartate aminotransferase, alanine aminotransferase, gamma glutamyl transferase, urea nitrogen, creatinine, total protein, albumin, globulin and A/G (albumin/globulin) ratio (calculated), glucose, total cholesterol, triglycerides, electrolytes (sodium, potassium, chloride), calcium, phosphorus, and sample quality. The urinalysis parameters include one or more of volume, color and appearance, specific gravity, pH, protein, glucose, bilirubin, ketones, blood, and urobilinogen.

For toxicokinetic evaluation, blood samples are also collected through the jugular vein (or other suitable vein) and processed for plasma to analyze tianeptine hemi-oxalate on Day 1 and Day 7 at post-dose collection intervals of 0.5, 1, 2, 4, 8 and 24 hours of Groups 2-5. A non-compartmental approach consistent with the oral route of administration is used for parameter estimation including assessing one or more of tₘₐₓ, Cₘₐₓ, Cₘₐₓ/Dose, AUCₜₗₐₛₜ, AUCₜₗₐₛₜ/Dose, AUC₍₀₋ₜ₎, AUC₍₀₋ₜ₎/Dose, tₗₐₛₜ, and R_{AUC}. All parameters are generated from tianeptine hemi-oxalate individual concentrations in plasma from Days 1 and 7 whenever practical.

Tissues are collected, weighed and preserved in 10% neutral buffered formalin for processing and evaluation (e.g., animal ID and macroscopic evaluation (e.g., abnormalities)). Tissues that are collected include one or more of artery (e.g., aorta), bone marrow smear, bone marrow (e.g., sternum), bone (e.g., femur and/or sternum), brain, epididymis(ides), esophagus, eye(s), gallbladder, ganglion (e.g., dorsal root and/or lumbar), gland(s)(e.g., adrenal, lacrimal, mammary, parathyroid, pituitary, prostate, salivary (e.g., parotid, sublingual and/or mandibular), and/or thyroid), gut-associated lymphoid tissue, heart, joint (e.g., femorotibial), kidney(s), large intestine (e.g., cecum, colon and/or rectum), liver, lung(s), lymph node(s)(e.g., mandibular and/or mesenteric), muscle (e.g., skeletal), nerve (e.g., optic, sciatic and/or tibial), ovary(ies), oviduct(s), pancreas, skin, small intestine (e.g., duodenum, ileum, and/or jejunum), spinal cord (e.g., cervical, thoracic, and/or lumbar), spleen, stomach, testis(es), thymus, tongue, trachea, ureter, urinary bladder, uterus, cervix, and vagina. Bone marrow smears are collected from the femur at scheduled and unscheduled necropsies for possible examination. Smears are not collected from animals that are found dead or from animals that are euthanized moribund and then stored in the refrigerator prior to necropsy. Bone marrow smears are allowed to air dry and are not fixed in formalin. Eyes and optic nerves are preserved in Davidson's fixative. Testes and epididymides are preserved in modified Davidson's fixative.

A 56-day study with 28-day recovery period is performed to evaluate toxicity, toxicokinetic characteristics and reversibility of any findings after oral administration tianeptine hemi-oxalate or tianeptine hemi-oxalate in the presence of naloxone HCl dihydrate to beagle dogs. Dosing formulations are prepared under yellow lighting and adjusted for pH at appropriate concentrations to meet dose level requirements. The control formulation (vehicle) is 20% Captisol in deionized water with a pH between about 7.6-7.8. Tianeptine hemi-oxalate or tianeptine hemi-oxalate in combination with naloxone HCl dihydrate is added gradually into the vehicle to achieve a suspension at a target concentration and volume with a final pH between about 7.6-7.8. The dose levels and experimental groups are described in Table 7. The compositions (e.g., tianeptine hemi-oxalate with or without naloxone HCl dihydrate) or vehicle control are administered by oral gavage once per day for up to 56 days. The dose volume is based on the most recent body weight measurement. The values in ( ) are the equivalent salt dose levels/concentrations of the free base (tianeptine).

Blood and urine samples are collected twice during Pretest (all animals) and prior to terminal and recovery necropsies (Groups 1-5) to assess clinical pathology (i.e., hematology, coagulation, clinical chemistry and urinalysis). Blood samples are collected from the jugular vein (or other suitable vein). The order of bleeding is alternating (1 animal per sex from each dose group, then repeating) to reduce handling and time biases, unless concurrent with other timed functions. Urine samples are collected using steel pans placed under the cages for at least 16 hours. The hematology parameters include one or more of red blood cell count, hemoglobin concentration, hematocrit, mean corpuscular volume, red blood cell distribution width, mean corpuscular, hemoglobin concentration, mean corpuscular hemoglobin, reticulocyte count (absolute and percent), platelet count, white blood cell count, neutrophil count (absolute and percent), lymphocyte count (absolute and percent), monocyte count (absolute and percent), eosinophil count (absolute and percent), basophil count (absolute and percent), large unstained cells (absolute and percent), other cells (as appropriate), and blood smear (preserve and stain). The coagulation parameters include one or more of prothrombin time, activated partial thromboplastin time, fibrinogen, and sample quality. The clinical chemistry parameters include one or more of alkaline phosphatase, total bilirubin (when total bilirubin >1 mg/dL, indirect and direct bilirubin will also be measured), aspartate aminotransferase, alanine aminotransferase, gamma glutamyl transferase, urea nitrogen, creatinine, total protein, albumin, globulin (calculated), albumin/globulin ratio (calculated), glucose, total cholesterol, triglycerides, electrolytes (sodium, potassium, chloride), calcium, phosphorus, and sample quality. The urinalysis parameters include one or more of volume, color, appearance, specific gravity, pH, protein, glucose, bilirubin, ketones, blood and urobilinogen.

For toxicokinetic evaluation, blood samples from Groups 1-5 are also collected through the jugular vein (or other suitable vein) and processed for plasma to analyze tianeptine hemi-oxalate and naloxone HCl dihydrate on Day 1 at post-dose collection intervals at 0.5, 1, 2, 4, 8 and 24 hours and on Day 56 at 0 hour (pre-dose) and post-dose collection intervals at 0.5, 1, 2, 4, 8 and 24 hours. A non-compartmental approach consistent with the oral route of administration is used for parameter estimation including assessing one or more of tₘₐₓ, Cₘₐₓ, Cₘₐₓ/Dose, AUCₜₗₐₛₜ, AUCₜₗₐₛₜ/Dose, AUC₍₀₋ₜ₎, AUC₍₀₋ₜ₎/Dose, tₗₐₛₜ, and R_{AUC}. All parameters are generated from tianeptine hemi-oxalate and naloxone HCl dihydrate (Group 5 only) individual concentrations in plasma from Days 1 and 56 whenever practical.

Tissues are collected from Groups 1-5, weighed and preserved in 10% neutral buffered formalin for histology processing and evaluation (e.g., animal ID, microscopic evaluation, and macroscopic evaluation (e.g., abnormalities)). Tissues that are collected include one or more of artery (e.g., aorta), bone marrow smear, bone marrow (e.g., sternum), bone (e.g., femur and/or sternum), brain, epididymis(ides), esophagus, eye(s), gallbladder, ganglion (e.g., dorsal root and/or lumbar), gland(s) (e.g., adrenal, lacrimal, mammary, parathyroid, pituitary, prostate, salivary (e.g., parotid, sublingual and/or mandibular), and/or thyroid), gut-associated lymphoid tissue, heart, joint (e.g., femorotibial), kidney(s), large intestine (e.g., cecum, colon and/or rectum), liver, lung(s), lymph node(s)(e.g., mandibular and/or mesenteric), muscle (e.g., skeletal), nerve (e.g., optic, sciatic and/or tibial), ovary(ies), oviduct(s), pancreas, skin, small intestine (e.g., duodenum, ileum, and/or jejunum), spinal cord (e.g., cervical, thoracic, and/or lumbar), spleen, stomach, testis(es), thymus, tongue, trachea, ureter, urinary bladder, uterus, cervix, and vagina. Bone marrow smears are collected from the femur at scheduled and unscheduled necropsies for possible examination. Smears are not collected from animals that are found dead or from animals that are euthanized moribund and then stored in the refrigerator prior to necropsy. Bone marrow smears are allowed to air dry and are not fixed in formalin. Eyes and optic nerves are preserved in Davidson's fixative. Testes and epididymides are preserved in modified Davidson's fixative.

TABLE 5
Phase A—Maximum tolerated dose study

| | | | | Dose | Dose | Main Study | |
|---|---|---|---|---|---|---|---|
| | | Day | Dose | Vol- | Concen- | Number | Number |
| | | of | Level | ume | tration | of | of |
| | Com- | Dos- | (mL/ | (mL/ | (mg/ | Male | Female |
| Group | pound | ing | kg) | kg) | mL) | Rats | Rats |
| 1 | Tianeptine | 1 | 5 (5.5) | 5 | 1 (1.1) | 1* | 1* |
| | hemi- | | | | | | |
| | oxalate | | | | | | |
| | Tianeptine | 5 | 10 (11) | 5 | 2 (2.2) | | |
| | hemi- | | | | | | |
| | oxalate | | | | | | |
| | Tianeptine | 8 | 30 (33) | 5 | 6 (6.6) | | |
| | hemi- | | | | | | |
| | oxalate | | | | | | |
| | Tianeptine | 12 | 90 (99) | 5 | 18 (19.8) | | |
| | hemi- | | | | | | |
| | oxalate | | | | | | |

*After a 3-4 day washout period, the same animals will be administered up to three additional doses of tianeptine hemi-oxalate, if warranted. The proposed dose levels may be increased or decreased based on the response to the preceding dose level(s) until the dose-limiting toxicity is observed (MTD). Doses will not exceed 1000 mg/kg (limit dose).

TABLE 6
Phase B—7 Day toxicity and toxicokinetic study

| | | Dose | | Dose | | Number |
|---|---|---|---|---|---|---|
| | | Level | Dose | Concen- | Number | of |
| | | (mg/ | Volume | tration | of Male | Female |
| Group | Compound | kg/day) | (mL/kg) | (mg/mL) | Rats | Rats |
| 2 | Vehicle | 0 | 5 | 0 | 1 | 1 |
| 3 | Tianeptine | 5 | 5 | 1 (1.1) | 1 | 1 |
| | hemi-oxalate | | | | | |
| 4 | Tianeptine | 10 | 5 | 2 (2.2) | 1 | 1 |
| | hemi-oxalate | | | | | |
| 5 | Tianeptine | 30 | 5 | 6 (6.6) | 1 | 1 |
| | hemi-oxalate | | | | | |

TABLE 7
56-day study with 28-day recovery period experimental design with beagle dogs

| | | Dose | | Dose | Main Study | | Recovery Study | |
|---|---|---|---|---|---|---|---|---|
| | | Level | Dose | Concen- | Number | Number of | Number | Number of |
| | | (mg/kg/ | Volume | tration | of Male | Female | of Male | Female |
| Group | Compound | day) | (mL/kg) | (mg/mL) | Rats | Rats | Rats | Rats |
| 1 | Vehicle | 0 | 10 | 0 | 4 | 4 | 2 | 2 |
| | Control | | | | | | | |
| 2 | Tianeptine | 5 (5.5) | 10 | 1 (1.1) | 4 | 4 | 0 | 0 |
| | hemi- | | | | | | | |
| | oxalate | | | | | | | |
| 3 | Tianeptine | 10 (11) | 10 | 2 (2.2) | 4 | 4 | 0 | 0 |
| | hemi- | | | | | | | |
| | oxalate | | | | | | | |
| 4* | Tianeptine | 15 (16.5) | 10 | 3 (3.3) | 4 | 4 | 2 | 2 |
| | hemi- | | | | | | | |
| | oxalate | | | | | | | |
| 5* | Tianeptine | 15 | 10 | 3 | 4 | 4 | 2 | 2 |
| | hemi- | (16.5) + | | (3.3) + | | | | |
| | oxalate + | 0.38 | | 0.08 | | | | |
| | Naloxone | (0.46) | | (0.09) | | | | |
| | HCl | | | | | | | |
| | dihydrate | | | | | | | |

*Groups 4 and 5 receive a dose holiday starting on Days 5 (females) and 6 (males) due to clinical signs. The dose level is reduced when dosing resumes on Day 12 (females) and 13 (males).

### Example 3. Major Depressive Disorder Clinical Study

A Phase 2b, 6-week, randomized, double-blind, placebo-controlled, parallel-group, multicenter study is performed to evaluate the effects of the controlled release formulation of tianeptine hemi-oxalate and naloxone hydrochloride dihydrate on symptoms of depression in adult patients (ages 18-65) with major depressive disorder (MDD). Subjects ages 18 to 65 years (inclusive) are consented and screened (Visit 1) for the study until approximately 260 patients are randomized. Randomization is performed in a 1:1 ratio to one of 2 treatment arms (placebo or controlled release formulation of tianeptine hemi-oxalate and naloxone hydrochloride dihydrate daily) on subjects meeting DSM-5 diagnostic criteria for MDD in a current major depressive episode (MDE) (moderate or severe) as assessed by the Mini International Neuropsychiatric Interview, Version 7.0.2 (M.I.N.I17.0.2) at screening. The controlled release formulation of tianeptine hemi-oxalate and naloxone hydrochloride dihydrate dose for all eligible patients is 39.4 mg once a day (QD), initiated at Visit 2 (baseline). All subjects return to the study site for an off-drug safety follow-up visit approximately 2 weeks after discontinuation of the study drug. Overall duration in the study for each patient is up to 13 weeks.

The primary objective of the study is to evaluate the efficacy, tolerability and safety of controlled release formulation of tianeptine hemi-oxalate and naloxone hydrochloride dihydrate monotherapy in patients with MDD. The primary efficacy endpoint is the change from Baseline to Week 6 on the Montgomery-Åsberg Depression Rating Scale (MADRS) total score. The secondary objectives of the study include: 1. To evaluate the effects of the controlled release formulation of tianeptine hemi-oxalate and naloxone hydrochloride dihydrate versus placebo on clinician-rated disease severity, assessed by the Clinical Global Impression-Severity scale (CGI-S), -Improvement scale (CGI-I), and -Efficacy Index (CGI-EI); 2. To evaluate the effects of the controlled release formulation of tianeptine hemi-oxalate and naloxone hydrochloride dihydrate versus placebo on subject-rated disease severity, assessed by the Patient Global Impression Severity (PGI-S) and Improvement (PGI-I) scales; and 3. To evaluate the safety and tolerability of the controlled release formulation of tianeptine hemi-oxalate and naloxone hydrochloride dihydrate versus placebo in adult patients with MDD. Exploratory objectives include: 1. To explore responder rates of the controlled release formulation of tianeptine hemi-oxalate and naloxone hydrochloride dihydrate versus placebo, assessed by improvement in MADRS scores (30%, 240/o, 250%) and CGI-I (much or very much improved) from baseline to endpoint; 2. To explore the effect of the controlled release formulation of tianeptine hemi-oxalate and naloxone hydrochloride dihydrate on symptoms of anxiety, assessed by the Hamilton Anxiety Rating Scale (HARS); and 3. To explore the effect of the controlled release formulation of tianeptine hemi-oxalate and naloxone hydrochloride dihydrate on patient psychosocial function, assessed by the Sheehan Disability Scale (SDS) questionnaire.

### Example 4. Naloxone Blocks Toxicity of Tianeptine from Oral Abuse

In a chronic 56-day toxicity study in rats at a dose of 90 mg/kg/day tianeptine hemi-oxalate by oral gavage, 14 of 44 rats did not survive the dosing regimen (32% mortality), by the end of the in-life portion of the study. In a separate cohort, rats were administered an identical dose of 90 mg/kg/day tianeptine hemi-oxalate, combined with 2.28 mg/kg/day naloxone HCl (ratio of tianeptine oxalate to naloxone HCl=39.4:1), all the rats survived. The oral administration of naloxone at a dose of 2.28 mg/kg/day offers a survival benefit when administered in combination with a dose of 90 mg/kg/day of tianeptine. Naloxone has the potential to block toxicity of tianeptine or a pharmaceutically acceptable salt thereof or a polymorph thereof from oral abuse.

What is claimed is:

1. A composition comprising:
tianeptine or a pharmaceutically acceptable salt thereof or polymorphs of one or both; and
naloxone or a pharmaceutically acceptable salt thereof.

2. The composition of claim 1, wherein the pharmaceutically acceptable salt of the tianeptine is tianeptine oxalate or tianeptine sodium or polymorphs thereof.

3. The composition of claim 2, wherein the pharmaceutically acceptable salt of tianeptine is tianeptine oxalate or polymorphs thereof.

4. The composition of claim 3, wherein the tianeptine oxalate is an anhydrous crystalline hemi-oxalate salt, an anhydrous crystalline mono-oxalate salt, a mixture thereof or polymorphs of any of them.

5. The composition of claim 4, wherein the composition comprises two or more layers of tianeptine oxalate, each layer comprising one or more of the anhydrous crystalline hemi-oxalate salt, the anhydrous crystalline mono-oxalate salt, a mixture thereof or polymorphs of any of them.

6. The composition of claim 4, wherein the tianeptine oxalate is the anhydrous crystalline hemi-oxalate salt or polymorphs thereof.

7. The composition of claim 6, wherein the tianeptine oxalate salt is an anhydrous crystalline hemi-oxalate salt that exhibits an X-ray diffraction pattern (XRPD) comprising at least one peak selected from the group consisting of 8.2, 8.6, 9.1, and 9.5 degrees 2θ±0.3 degrees 2θ.

8. The composition of claim 7, wherein the anhydrous crystalline hemi-oxalate salt exhibits an XRPD pattern comprising at least one additional peak selected from the group consisting of 4.5, 11.5, 14.2, 15.2, 15.8, 16.2, 19.2, 22.1, 23.9, 26.9, and 27.4 degrees 2θ±0.3 degrees 2θ.

9. The composition of claim 4, wherein the tianeptine oxalate is an anhydrous crystalline mono-oxalate salt or polymorphs thereof.

10. The composition of claim 9, wherein the tianeptine oxalate salt is an anhydrous crystalline mono-oxalate salt that exhibits an X-ray diffraction pattern (XRPD) comprising at least one peak selected from the group consisting of 10.1 and 10.5 degrees 2θ±0.3 degrees 2θ.

11. The composition of claim 10, wherein the anhydrous crystalline mono-oxalate salt exhibits an XRPD pattern comprising at least one additional peak selected from the group consisting of 7.5, 8.3, 11.9, 14.7, 16.2, 16.3, 17.9, 18.7, 21.0, 21.7, and 22.1 degrees 2θ±0.3 degrees 2θ.

12. The composition of claim 4, wherein the tianeptine oxalate is the mixture of the anhydrous crystalline hemi-oxalate salt and the anhydrous crystalline mono-oxalate salt or polymorphs thereof.

13. The composition of claim 12, wherein the tianeptine oxalate is the mixture of the anhydrous crystalline hemi-oxalate salt and the anhydrous crystalline mono-oxalate salt that exhibits an X-ray diffraction pattern (XRPD) comprising at least one peak selected from the group consist of 10.1 and 10.5 degrees 2θ±0.3 degrees 2θ.

14. The composition of claim 13, wherein the mixture exhibits an XRPD pattern further comprising at least one additional peak selected from the group consisting of 7.5, 8.3, 11.9, 14.7, 16.2, 16.3, 17.9, 18.7, 21.0, 21.7, and 22.1 degrees 2θ±0.3 degrees 2θ.

15. The composition of claim 1, wherein the pharmaceutically acceptable salt of naloxone is an acid salt.

16. The composition of claim 15, wherein the naloxone acid salt is naloxone hydrochloride.

17. The composition of claim 16, wherein the naloxone hydrochloride is naloxone hydrochloride dihydrate.

18. The composition of claim 1, wherein the tianeptine, the pharmaceutically acceptable salt thereof or the polymorph of one or both is present in an amount between 1% w/w to 30% w/w, between 5% w/w to 25% w/w, or between 10% w/w to 20% w/w.

19. The composition of claim 18, wherein the tianeptine, the pharmaceutically acceptable salt thereof or the polymorph of one or both is present in an amount equivalent to about 14.28% w/w of the tianeptine free base.

20. The composition of claim 18, wherein the pharmaceutically acceptable salt of tianeptine is an anhydrous crystalline hemi-oxalate salt or a polymorph thereof.

21. The composition of claim 20, wherein the anhydrous crystalline hemi-oxalate salt or the polymorph thereof is an oxalate salt.

22. The composition of claim 21, wherein the tianeptine hemi-oxalate salt is present in an amount of about 15.76% w/w.

23. The composition of claim 1, wherein the naloxone or the pharmaceutically acceptable salt thereof is present in an amount between 0.01% w/w to 5% w/w or between 0.1% w/w to 2% w/w.

24. The composition of claim 23, wherein the naloxone or the pharmaceutically acceptable salt thereof is present in an amount of less than 1% w/w or less than 0.5% w/w.

25. The composition of claim 24, wherein the naloxone or the pharmaceutically acceptable salt thereof is present in an amount equivalent to about 0.40% w/w of the naloxone free base.

26. The composition of claim 24, wherein the pharmaceutically acceptable salt of naloxone is naloxone hydrochloride.

27. The composition of claim 26, wherein the naloxone hydrochloride is naloxone hydrochloride dihydrate.

28. The composition of claim 27, wherein the naloxone hydrochloride dihydrate is present in an amount of about 0.49% w/w.

29. The composition of claim 1, wherein the tianeptine, the pharmaceutically acceptable salt there of or the polymorph of one or both is present in an amount between 0.1 mg to 60 mg, between 1 mg to 50 mg, or between 10 mg to 40 mg.

30. The composition of claim 29, wherein the tianeptine, the pharmaceutically acceptable salt thereof or the polymorph of one or both is present in the composition in an amount of 50 mg or less, 45 mg or less, or 40 mg or less.

31. The composition of claim 30, wherein the tianeptine, the pharmaceutically acceptable salt thereof or the polymorph of one or both is present in an amount equivalent to about 35.7 mg of the tianeptine free base.

32. The composition of claim 30, wherein the pharmaceutically acceptable salt of tianeptine is tianeptine oxalate or polymorphs thereof.

33. The composition of claim 32, wherein the tianeptine oxalate is an anhydrous crystalline hemi-oxalate salt or polymorph thereof.

34. The composition of claim 33, wherein the anhydrous crystalline tianeptine hemi-oxalate salt or polymorph thereof is present in an amount of about 39.4 mg.

35. The composition of claim 1, wherein the naloxone or the pharmaceutically acceptable salt thereof is present in an amount between 0.01 mg to 10 mg, between 0.1 mg to 5 mg, or between 0.5 mg to 2 mg.

36. The composition of claim 35, wherein the naloxone or the pharmaceutically acceptable salt thereof is present in an amount of 5 mg or less, or 2 mg or less.

37. The composition of claim 36, wherein the naloxone or the pharmaceutically acceptable salt thereof is present in an amount equivalent to about 1 mg of the naloxone free base.

38. The composition of claim 36, wherein the pharmaceutically acceptable salt of naloxone is naloxone hydrochloride.

39. The composition of claim 38, wherein the naloxone hydrochloride is naloxone hydrochloride dihydrate.

40. The composition of claim 39, wherein the naloxone hydrochloride dihydrate is present in an amount of about 1.22 mg.

41. The composition of any one of claims 1-40, wherein the composition further comprises one or more excipients.

42. The composition of claim 41, wherein the excipient comprises a filler, a controlled release polymer, a lubricant, a glidant, a pH adjusting agent, or a buffer.

43. The composition of claim 42, wherein the filler is selected from the group consisting of microcrystalline cellulose, gelatin, sucrose, starch, acacia, tragacanth, alginic acid, cellulose, methyl cellulose, ethyl cellulose, hydroxypropyl methyl cellulose, hydroxypropyl cellulose, sodium carboxymethyl cellulose, polyvinyl pyrrolidone, polyethylene glycol, polyvinyl pyrrolidone, lactose monohydrate and cross-linked polyvinyl pyrrolidone.

44. The composition of claim 43, wherein the filler is microcrystalline cellulose or lactose monohydrate.

45. The composition of claim 43 or 44, wherein the filler is present in an amount between 0.5% w/w to 50% w/w.

46. The composition of claim 42, wherein the controlled release polymer is selected from the group consisting of hydroxypropyl methylcellulose, hydroxypropyl cellulose, polyvinyl pyrrolidone, carboxymethyl cellulose, methylcellulose, hydroxyethyl cellulose, ethyl hydroxyethyl cellulose, sodium carboxymethyl cellulose, sodium alginate, xanthan gum, carrageenan, chitosan, guar gum, pectin, polyethylene oxide, ethylcellulose, hypromellose acetate succinate, cellulose acetate and cellulose acetate propionate.

47. The composition of claim 46, wherein the controlled release polymer is hydroxypropyl methylcellulose.

48. The composition of claim 46 or 47, wherein the controlled release polymer is present in an amount between 5% w/w to 30% w/w.

49. The composition of claim 42, wherein the lubricant is selected from the group consisting of magnesium stearate, calcium stearate, zinc stearate, stearic acid, myristic acid, palmitic acid, sodium stearyl fumarate, boric acid, carbowax (PEG) 4000/6000, sodium oleate, sodium benzoate, sodium acetate, sodium lauryl sulfate, magnesium lauryl sulfate, glyceride esters, glyceryl monostearate, glyceryl tribehenate, glyceryl dibehenate, sugar esters, sorbitan monostearate, sucrose monopalmitate, waxes, sterowet, glyceryl behenate, liquid paraffin and talc.

50. The composition of claim 49 wherein the lubricant is magnesium stearate.

51. The composition of claim 49 or 50, wherein the lubricant is present in an amount between 0.1% w/w to 5% w/w.

52. The composition of claim 42, wherein the glidant is selected from the group consisting of starch, cornstarch, silica derivatives, fumed silica, colloidal silicon dioxide, hydrophilic fumed silica, syloid, pyrogenic silica, ascorbyl palmitate, calcium palmitate, magnesium stearate, talc and hydrated sodium sulfoaluminate.

53. The composition of claim 52, wherein the glidant is colloidal silicon dioxide.

54. The composition of claim 52 or 53 wherein the glidant is present in an amount between 0.1% w/w to 2% w/w.

55. The composition of claim 42, wherein the pH adjusting agent is selected from the group consisting of citric acid monohydrate, trisodium citrate dihydrate, sodium acetate, acetic acid, tartaric acid, sodium hydrogen carbonate, potassium citrate monohydrate, sodium citrate, tromethamine (Tris), sodium hydroxide, sodium phosphate, disodium hydrogen phosphate, sodium bisphosphate, boric acid, sodium borate, benzoic acid, sodium benzoate, aspartic acid, and maleic acid.

56. The composition of claim 55, wherein the pH adjusting agent is citric acid monohydrate, trisodium citrate dihydrate, or a combination thereof.

57. The composition of claim 55 or 56, wherein the pH adjusting agent is present in an amount between 0.1% w/w to 2% w/w.

58. The composition of claim 42, wherein the buffer is selected from the group consisting of trisodium citrate dihydrate, citric acid, boric acid, sodium citrate, potassium bitartrate, calcium acetate, ammonium phosphate, calcium chloride, citric acid salts, lactic acid, magnesium trisilicate, phosphoric acid, potassium, potassium citrate, potassium phosphate, sodium acetate, sodium borate, sodium lactate, succinic acid, and monothioglycerol.

59. The composition of claim 58, wherein the buffer is trisodium citrate dihydrate.

60. The composition of claim 58 or 59, wherein the buffer is present in an amount between 0.1% w/w to 2% w/w.

61. The composition of any one of claims 1-60, wherein the composition further comprises a pharmaceutically acceptable carrier.

62. The composition of any one of claims 1-61, wherein the composition is in the form of a tablet, a mini-tablet, a chewable tablet, an orodispersible tablet, a dissolvable tablet, a scored tablet, a coated tablet, a suppository, or a granule.

63. The composition of claim 62, wherein the composition is in the form of a tablet.

64. The composition of claim 63, wherein the tablet is characterized by a hardness of greater than 10 Kp.

65. The composition of claim 63 or 64, wherein the tablet is characterized by a tensile strength of greater than 2 MPa.

66. The composition of any one of claims 63-65, wherein the tablet is formulated so as to be capable of immediate release, controlled release, sustained release, extended release, or slow release of one or both of the tianeptine, the pharmaceutically acceptable salt thereof or the polymorph of one or both and the naloxone or the pharmaceutically salt thereof.

67. The composition of claim 66, wherein the tablet is formulated so as to be capable of controlled release of the tianeptine, the pharmaceutically acceptable salt thereof or the polymorph of one or both and the naloxone or the pharmaceutically acceptable salt thereof.

68. The composition of claim 67, wherein the tablet is formulated so as to be capable of controlled release of the pharmaceutically acceptable salt of tianeptine or the polymorph thereof and the pharmaceutically acceptable salt of naloxone.

69. The composition of claim 68, wherein the pharmaceutically acceptable salt of tianeptine is tianeptine oxalate or polymorphs thereof and the pharmaceutically acceptable salt of naloxone is naloxone hydrochloride.

70. The composition of claim 69, wherein the tianeptine oxalate is an anhydrous crystalline hemi-oxalate salt and wherein the naloxone hydrochloride is naloxone hydrochloride dihydrate.

71. The composition of any one of claims 1-70, wherein the composition prevents or reduces the potential for abuse of the tianeptine, the pharmaceutically acceptable salt or the polymorph.

72. A method of preventing and/or treating major depressive disorder (MDD) or symptoms associated therewith, comprising administering to a subject in need thereof or at risk thereof, the composition of any one of claims 1-71.

73. The method of claim 72, wherein the composition is administered one or more times daily.

74. The method of claim 73, wherein the composition is administered once daily.

75. The method of any one of claims 72-74, wherein the composition is administered orally, sublingually, buccally, palatially, rectally, or vaginally.

76. The method of claim 75, wherein the composition is administered orally.

77. A method of manufacturing the composition of any one of claims 1-71, wherein the naloxone or the pharmaceutically acceptable salt thereof is mixed with the pH adjusting agent prior to its mixing with the tianeptine, the pharmaceutically acceptable salt thereof or the polymorph of one or both, and the one or more excipients.

78. The method of claim 77, wherein the naloxone or the pharmaceutically acceptable salt thereof before mixing is comprised of particles that are less than 20 μm.

79. The method of claim 77 or 78, wherein the composition is compressed to form a tablet.

80. The method of claim 79, wherein the tablet is compressed to a hardness of greater than 10 Kp.

81. The method of claim 79 or 80, wherein the tablet is compressed to a tensile strength of greater than 2 MPa.
